SEQUENCE LISTING (13 sequences)

SEQ ID NO: 1 - DNA, 20 bp, Artificial Sequence
  Oligonucleotide probe
aacacaacacaacacaacac

SEQ ID NO: 2 - DNA, 15 bp, Artificial Sequence
  Oligonucleotide probe
acgggaccagtacgg

SEQ ID NO: 3 - DNA, 20 bp, Artificial Sequence
  Oligonucleotide probe
ctcaagaagatacaaggaca

SEQ ID NO: 4 - DNA, 20 bp, Artificial Sequence
  Oligonucleotide probe
ccagtgcagaagaaaatcaa

SEQ ID NO: 5 - DNA, 19 bp, Artificial Sequence
  Oligonucleotide probe
gcccatagactgaaataga

SEQ ID NO: 6 - DNA, 22 bp, Artificial Sequence
  Oligonucleotide probe
ggacgtggaaaaggtggcaaag

SEQ ID NO: 7 - DNA, 22 bp, Artificial Sequence
  Oligonucleotide probe
aagcgctcgaccatcaccagtc

SEQ ID NO: 8 - DNA, 22 bp, Artificial Sequence
  Oligonucleotide probe
aaaaagacggtgaagaaagcat

SEQ ID NO: 9 - DNA, 15 bp, Artificial Sequence
  Oligonucleotide probe
acgggaccagtacgg

SEQ ID NO: 10 - DNA, 17 bp, Artificial Sequence
  Oligonucleotide probe
gtgagcggataacaatt

SEQ ID NO: 11 - DNA, 17 bp, Artificial Sequence
  Oligonucleotide probe
atgtggaattgtgagcg

SEQ ID NO: 12 - DNA, 20 bp, Artificial Sequence
  Oligonucleotide probe
aacacaacacaacacaacac

SEQ ID NO: 13 - DNA, 20 bp, Artificial Sequence
  Oligonucleotide probe
aatagaatagaatagaatag

(19) United States
(12) Patent Application Publication
(10) Pub. No.: US 2018/0292318 A1
(43) Pub. Date: Oct. 11, 2018

(54) High-Throughput In Situ Hybridization

(71) Applicant: President and Fellows of Harvard College, Cambridge, MA (US)

(72) Inventors: Chao-ting Wu, Brookline, MA (US); Brian Beliveau, Cambridge, MA (US)

(21) Appl. No.: 15/726,913

(22) Filed: Oct. 6, 2017

Related U.S. Application Data
(63) Continuation of application No. 13/398,945, filed on Feb. 17, 2012, now abandoned.
(60) Provisional application No. 61/443,904, filed on Feb. 17, 2011.

(51) Int. Cl. G01N 21/64; C12Q 1/6841

(52) U.S. Cl. CPC G01N 21/6428; C12Q 2563/107; C12Q 1/6841; G01N 21/6452

(57) ABSTRACT

Novel methods and compositions for providing high-throughput fluorescence in situ hybridization (FISH) are provided.

### RELATED APPLICATION DATA

This application is a continuation application which claims priority to U.S. patent application Ser. No. 13/398,945, filed on Feb. 17, 2012, which claims the benefit of U.S. Provisional Patent Application No. 61/443,904, filed on Feb. 17, 2011 each of which are hereby incorporated by reference in their entireties.

### STATEMENT OF GOVERNMENT INTERESTS

This invention was made with Government support under the National Institutes of Health grant number GM085169-01A1. The Government has certain rights in the invention.

### FIELD

Embodiments of the present invention relate in general to high-throughput in situ hybridization such as, e.g., fluorescence in situ hybridization (FISH).

### BACKGROUND

Fluorescence in situ hybridization (FISH) is a cytogenetic technique that is used to detect and localize the presence or absence of specific DNA sequences, e.g., DNA sequences on chromosomes. FISH uses fluorescent probes that bind to only those parts of the chromosome with which they show a high degree of sequence similarity. Fluorescence microscopy can be used to determine where the fluorescent probe bound to the chromosomes. FISH is often used for finding specific features in DNA for use in genetic counseling, medicine and species identification. FISH can also be used to detect and localize specific mRNAs within tissue samples. In this context, it can help define the spatial-temporal patterns of gene expression within cells and tissues.

Recently, methods of performing parallel (i.e., high-throughput) FISH using locked nucleic acid (LNA) probes have been developed. The cost of using LNAs is prohibitive, however, and effectively restricts wide-spread use of high-throughput FISH using LNAs. Accordingly, less expensive methods of performing high-throughput FISH are needed.

### SUMMARY

The present invention is based in part on the discovery that probes and/or oligonucleotide paints can be used to perform high-throughput in situ hybridization, e.g., FISH, effecting massive savings in cost. The present invention is further based on the discovery of a mechanical means of reducing the amount of reagents needed to perform high-throughput FISH assays.

Accordingly, a first method for performing FISH is provided. The method includes the steps of providing a biological sample, contacting the biological sample with an oligonucleotide paint having a fluorescent label attached thereto, allowing the oligonucleotide paint to bind to the biological sample, and detecting binding of the oligonucleotide paint. In certain aspects, a plurality of oligonucleotide paints is used. In certain aspects, the oligonucleotide paint crosses a cell membrane and/or a nuclear membrane. In other aspects, the oligonucleotide paint binds to the biological sample by hybridizing to a target sequence (e.g., a nucleic acid sequence (e.g., a genomic nucleic acid sequence)). In yet other aspects, a plurality of biological samples are provided on a multi-well plate (e.g., a 384-well plate). In still other aspects, a plurality of biological samples are provided on a separable multi-well apparatus having a well-forming component and a base component. In certain aspects, the well-forming component is removed from the separable multi-well apparatus, e.g., after the step of providing the sample or after the oligonucleotide paint binds to the sample. In other aspects, the steps of removing the well-forming component and contacting the base component with one or more reagents are performed between the steps of allowing and detecting.

A second method for performing FISH is provided. The method includes the steps of providing a biological sample, providing an oligonucleotide paint that lacks a 3′ primer sequence and has a fluorescent label attached thereto, contacting the biological sample with the oligonucleotide paint, allowing the oligonucleotide paint to bind to the biological sample, and detecting binding of the oligonucleotide paint. In certain aspects, the 3′ primer sequence is removed from the oligonucleotide paint by contacting the oligonucleotide paint with a nicking endonuclease. In yet other aspects, the oligonucleotide paint having the 3′ primer sequence removed binds the biological sample with a greater affinity when compared to an oligonucleotide paint having a 3′ primer sequence present.

A third method for performing FISH is provided. The method includes the steps of providing a biological sample, providing an oligonucleotide paint that lacks a 3′ primer sequence and a 5′ primer sequence and has a fluorescent label attached thereto, contacting the biological sample with the oligonucleotide paint, allowing the oligonucleotide paint to bind to the biological sample, and detecting binding of the oligonucleotide paint. In certain aspects, the 3′ and the 5′ primer sequences are removed from the oligonucleotide paint by contacting the oligonucleotide paint with a type IIS restriction enzyme. In certain aspects, the fluorescent label is attached to the oligonucleotide paint using terminal transferase. In yet other aspects, the oligonucleotide paint having the 3′ and 5′ primer sequences removed binds the biological sample with a greater affinity when compared to an oligonucleotide paint having 3′ and 5′ primer sequences present.

A fourth method for performing FISH is provided. The method includes the steps of providing a biological sample, contacting the biological sample with an enzyme that cleaves DNA, contacting the biological sample with an oligonucleotide paint having a fluorescent label bound thereto, allowing the oligonucleotide paint to bind to the biological sample, and detecting binding of the oligonucleotide paint. In certain aspects, the enzyme that cleaves DNA is one or both of a nuclease (e.g., DNase I and/or micrococcal nuclease) and a restriction enzyme. In other aspects, the oligonucleotide paint binds to the biological sample by hybridizing to genomic DNA.

A separable multi-well apparatus is provided. The separable multi-well apparatus includes a well-forming component, and a base component, wherein the well-forming component is aligned over the base component and wherein the well-forming component and the base component are separably attached to each other to form a separable multi-well apparatus. In certain aspects, the well-forming component and the base component are attached to each other by one or more of an adhesive, a magnetic force, a mechanical force and a sealant. In other aspects, the well-forming component comprises at least 8 wells, at least 500 wells, or 384 wells.

Further features and advantages of certain embodiments of the present invention will become more fully apparent in the following description of the embodiments and drawings thereof, and from the claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The foregoing and other features and advantages of the present invention will be more fully understood from the following detailed description of illustrative embodiments taken in conjunction with the accompanying drawings in which:

FIG. 1 depicts a plan view of a separable multi-well apparatus having a base component and a separable well-forming component according to one embodiment of the invention. The top portion of the figure depicts a base component and a separable well-forming component attached to form a separable multi-well apparatus. The bottom portion of the figure depicts a separated base component.

FIG. 2 schematically depicts a method according to the invention.

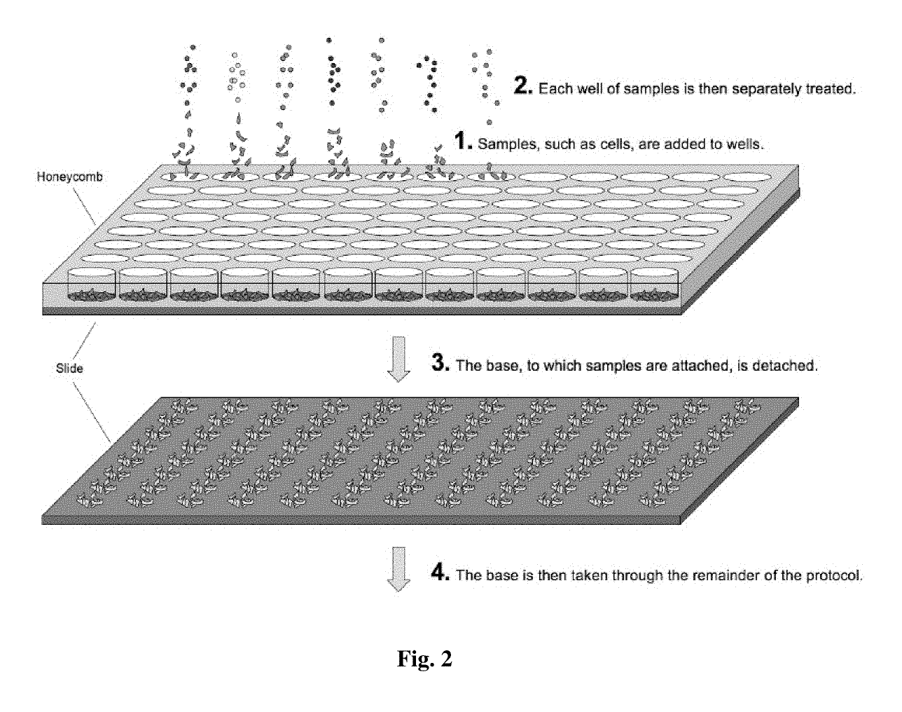

FIG. 3 schematically illustrates the use of a locked nucleic acid (LNA) high-throughput fluorescence in situ hybridization (FISH) screening. Kc₁₆₇ cells and double stranded (ds) RNA were combined in a 384-well plate, in which one well corresponded to one unique ds RNA. Cells were then incubated for 4-5 days, and then fixed with formaldehyde. LNA FISH was performed and the cells were imaged on an automated confocal microscope. The images were processed with automated software. The data was analyzed to identify ds RNA that increased the number of FISH signals per nucleus, an indicator of pairing.

FIG. 4 graphically compares standard FISH with high-throughput LNA FISH.

FIG. 5 depicts a gel showing that 60-mer oligonucleotides harvested from an array served as good templates for PCR amplification by six of seven primer pairs. Overall, 43 of 55 primer pairs worked.

FIG. 6 depicts signals obtained using a 60-bp single stranded (ss) probe targeting approximately 110 copies of a 32-bp target (panel A) and a 32-bp ss probe via 384-well LNA FISH (panel B). Images were contrast adjusted.

FIG. 7 schematically depicts one protocol for the synthesis of Oligopaints from array-derived oligonucleotides. Other protocols can be used as described herein.

FIG. 8 schematically depicts custom-designed paints that are generated via PCR amplification of genomic sequences synthesized on arrays.

FIG. 9 depicts one strategy by which the 24 chromosomes of the human genome are differentially colored with a base color consisting of a mix of five primary colors and a series of color-coded bands staggered along the p and q arms.

FIG. 10 schematically depicts a first strategy for removing primer sequences.

FIG. 11 schematically depicts a second strategy for removing primer sequences.

### DETAILED DESCRIPTION

One of the most time-consuming and difficult aspects of FISH methods known in the art at the time of filing is the awkwardness of using multi-well plates. Accordingly, the present invention is directed in part to the creation of separable multi-well apparatuses and methods that use them. The separable multi-well apparatuses described herein permit researchers to detach the base component (e.g., a slide) after the cells have adhered to the bottom of the wells and been treated with one or more reagents (e.g., RNAi, small molecules, test compounds, etc.). The detached base component can then be processed in parallel with many other base components, simplifying subsequent steps of any protocol and reducing the amounts of resources required. Separable multi-well plates advantageously reduce the amount of reagents needed for a variety of assays compared with the use of traditional multi-well plates.

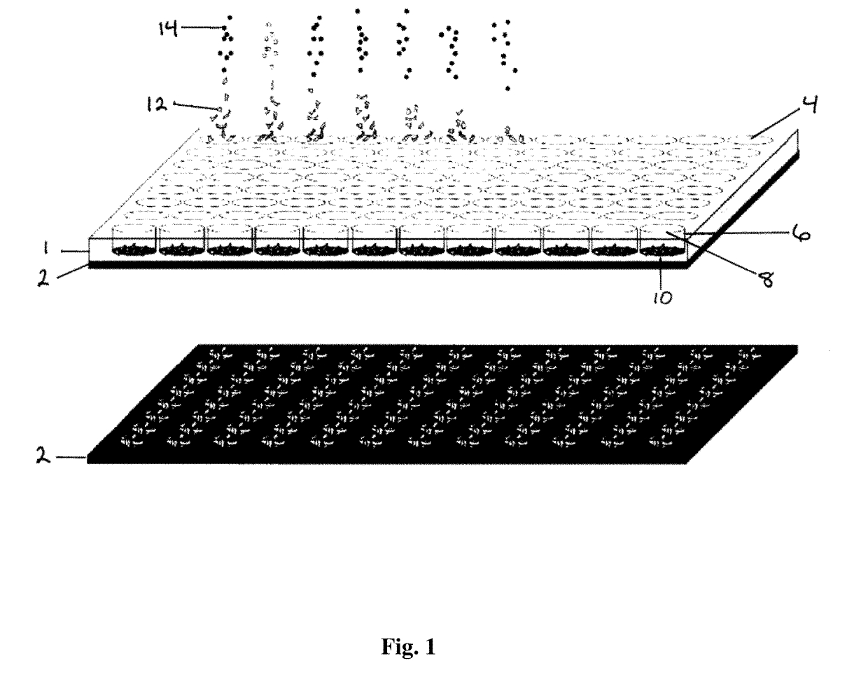

Depicted in FIG. 1 is one version of a separable multi-well apparatus of the present invention. This version of a separable multi-well apparatus includes two parts: well-forming component 1 and base component 2. When assembled, a separable multi-well apparatus contains a plurality of wells 4, each well having a side 6, a top edge 8 and a bottom 10. After separation of well-forming component 1 from base component 2, bottom 10 is no longer located within a well of well-forming component 1, and is instead located at a discrete location on base component 2.

A separable multi-well apparatus as described herein may contain any number of individual wells. In certain aspects, a multi-well apparatus will have about 2, 5, 10, 12, 16, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 384, 390, 400, 410, 420, 430, 440, 450, 460, 470, 480, 490, 500, 550, 600, 650, 700, 750, 800, 850, 900, 950, 1000, 2000, 3000, 4000, 5000, 6000, 7000, 8000, 9000, 10,000, 25,000, 50,000, 75,000, 100,000, 1,000,000 or more wells, or any values in between. Wells can be a variety of shapes, e.g., round, oval, square, rectangular, triangular, pentagonal, hexagonal, etc. A removable multi-well apparatus can be made from a variety of materials known in the art (such as those typically used to make traditional multi-well plates), including, but not limited to, glass, quartz, ceramic, plastic, polystyrene, methylstyrene, acrylic polymers, titanium, latex, sepharose, cellulose, nylon and the like and any combination thereof. In certain aspects, the well-forming component is made of the same material as the base component. In other aspects, the well-forming component is made of a different material than the base component.

According to certain exemplary embodiments of the invention, well-forming component 1 is reversibly attached to base component 2. Attaching can be achieved using a variety of methods and/or compositions. A separable multi-well apparatus is made using a variety of approaches (and any combinations thereof): 1) one or more adhesives (e.g., glues, gums, resins, drying adhesives, contact adhesives, hot adhesives, reactive adhesives, pressure sensitive adhesives and the like) are used to bind the base component to the well-forming component; 2) magnetic force is used to bind the base component to the well-forming component; 3) a mechanical force (e.g., clamps, staples, tape, straps, fasteners, folds and the like) is used hold the base component and the well-forming component together; and/or 4) a sealant is used to bind the base component and the well-forming component together. Detachment of the base component from the well-forming component is performed using a variety of approaches: 1) dissolution of the adhesive; 2) pulling the two magnetic components or one magnetic component and one metallic component apart or shutting down the magnetic force; 3) unclamping the apparatus; and/or 4) removing, dissolving or otherwise overcoming the sealing ability of the sealant. Other attaching and detaching approaches would be readily understood by one of ordinary skill in the art based on the disclosures provided herein and the knowledge in the art.

As used herein, the terms “bound” and “attached” refer to both covalent interactions and noncovalent interactions. A covalent interaction is a chemical linkage between two atoms or radicals formed by the sharing of a pair of electrons (i.e., a single bond), two pairs of electrons (i.e., a double bond) or three pairs of electrons (i.e., a triple bond). Covalent interactions are also known in the art as electron pair interactions or electron pair bonds. Noncovalent interactions include, but are not limited to, van der Waals interactions, hydrogen bonds, weak chemical bonds (i.e., via short-range noncovalent forces), hydrophobic interactions, ionic bonds and the like. A review of noncovalent interactions can be found in Alberts et al., in Molecular Biology of the Cell, 3d edition, Garland Publishing, 1994.

According to certain exemplary embodiments of the invention, a separable multi-well apparatus can be used to perform a variety of assays such as, e.g., FISH. In certain aspects, a separable multi-well apparatus can be used as follows: Step A, sample 12 (e.g., one or more cells, antibodies, oligonucleotides or the like) is added to well 4 and attaches to bottom 10; Step B, well 4 is contacted with reagent 14 (optionally, one or more agents); Step C, base component 2 is separated from well-forming component 1; and Step D, base component 2 is optionally contacted with one or more additional reagents (FIG. 1). After separation of well-forming component 1 from base component 2, sample 12 substantially remains attached to base component 2 at a discrete location.

Embodiments of the present invention are further directed to methods and compositions for performing FISH in parallel (e.g., high-throughput FISH) without the need for costly LNAs. In certain aspects, methods of using probes and/or oligonucleotide paints with high-throughput FISH are provided. In certain aspects, methods of performing high-throughput FISH with oligonucleotide paints using a multi-well plate (e.g., a traditional multi-well plate (e.g., a 384-well plate) or a separable multi-well apparatus) are provided. In certain aspects, methods of performing whole-genome screens using high-throughput FISH with oligonucleotide paints are provided.

Under certain circumstances, the presence of primer sequences at the both ends of the Oligopaint probes can be inhibitory to FISH. Accordingly, in certain aspects, methods of cleaving a primer sequence from one end of a probe are provided, greatly enhancing FISH signals.

Hybridization of nucleic acid probes can be inhibited by topological constraints of the interacting nucleic acids. For example, hybridization of a short oligonucleotide probe to a long double-stranded DNA molecule requires the ‘melting’ of the two strands of the double-stranded DNA, which may be impeded by topological constraints resulting from the unwinding of the two strands. Accordingly, in certain aspects, methods of releasing conformational restraints and, accordingly, increasing the affinity of a nucleic acid sequence to a target are provided. In certain aspects, conformational restraints are released and/or reduced by contacting the target with one or more nucleases, e.g., DNase 1, MNase, nicking endonucleases, cutting endonucleases and the like. In certain aspects, contact with one or more nucleases is performed after fixation of the target.

As used herein, the terms “probe,” “oligonucleotide paint” and “Oligopaint” refer to detectably labeled polynucleotides that have sequences complementary to an oligonucleotide sequence, e.g., a portion of a nucleic acid sequence, such as a DNA sequence. In certain aspects, a probe, an oligonucleotide paint or an Oligopaint contains sequences complementary to an oligonucleotide sequence of a particular chromosome or sub-chromosomal region of a particular chromosome. Probes and Oligopaints can be generated from synthetic probes and arrays that are, optionally, computationally patterned (rather than using natural DNA sequences and/or chromosomes as a template). Oligopaints are described in detail in U.S. Ser. No. 12/780,446, Filed May 14, 2010 (U.S. Publication No. 2010/0304994), and in the patent application corresponding to Attorney Docket Number 10498-00271, each of which is incorporated herein by reference in its entirety for all purposes.

As used herein, the terms “Oligopainted” and “Oligopainted region” refer to a target nucleotide sequence (e.g., a chromosome) or region of a target nucleotide sequence (e.g., a sub-chromosomal region), respectively, that has hybridized thereto one or more Oligopaints. Oligopaints can be used to label a target nucleotide sequence, e.g., chromosomes and sub-chromosomal regions of chromosomes during various phases of the cell cycle including, but not limited to, interphase, preprophase, prophase, prometaphase, metaphase, anaphase, telophase and cytokenesis.

As used herein, the term “chromosome” refers to the support for the genes carrying heredity in a living cell, including DNA, protein, RNA and other associated factors. The conventional international system for identifying and numbering the chromosomes of the human genome is used herein. The size of an individual chromosome may vary within a multi-chromosomal genome and from one genome to another. A chromosome can be obtained from any species. A chromosome can be obtained from an adult subject, a juvenile subject, an infant subject, from an unborn subject (e.g., from a fetus, e.g., via prenatal test such as amniocentesis, chorionic villus sampling, and the like or directly from the fetus, e.g., during a fetal surgery) from a biological sample (e.g., a biological tissue, fluid or cells (e.g., sputum, blood, blood cells, tissue or fine needle biopsy samples, urine, cerebrospinal fluid, peritoneal fluid, and pleural fluid, or cells therefrom) or from a cell culture sample (e.g., primary cells, immortalized cells, partially immortalized cells or the like). In certain exemplary embodiments, one or more chromosomes can be obtained from one or more genera including, but not limited to, Homo, Drosophila, Caenorhabiditis, Danio, Cyprinus, Equus, Canis, Ovis, Ocorynchus, Salmo, Bos, Sus, Gallus, Solanum, Triticum, Oryza, Zea, Hordeum, Musa, Avena, Populus, Brassica, Saccharum and the like.

As used herein, the term “chromosome banding” refers to differential staining of chromosomes resulting in a pattern of transverse bands of distinguishable (e.g., differently or alternately colored) regions, that is characteristic for the individual chromosome or chromosome region (i.e., the “banding pattern”). Conventional banding techniques include G-banding (Giemsa stain), Q-banding (Quinacrine mustard stain), R-banding (reverse-Giemsa), and C-banding (centromere banding).

As used herein, the term “karyotype” refers to the chromosome characteristics of an individual cell, cell line or genome of a given species, as defined by both the number and morphology of the chromosomes. Karyotype can refer to a variety of chromosomal rearrangements including, but not limited to, translocations, insertional translocations, inversions, deletions, duplications, transpositions, anueploidies, complex rearrangements, telomere loss and the like. Typically, the karyotype is presented as a systematized array of prophase or metaphase (or otherwise condensed) chromosomes from a photomicrograph or computer-generated image. Interphase chromosomes may also be examined.

As used herein, the terms “chromosomal aberration” or “chromosome abnormality” refer to a deviation between the structure of the subject chromosome or karyotype and a normal (i.e., non-aberrant) homologous chromosome or karyotype. The deviation may be of a single base pair or of many base pairs. The terms “normal” or “non-aberrant,” when referring to chromosomes or karyotypes, refer to the karyotype or banding pattern found in healthy individuals of a particular species and gender. Chromosome abnormalities can be numerical or structural in nature, and include, but are not limited to, aneuploidy, polyploidy, inversion, translocation, deletion, duplication and the like. Chromosome abnormalities may be correlated with the presence of a pathological condition or with a predisposition to developing a pathological condition.

Chromosome aberrations and/or abnormalities can also refer to changes that are not associated with a disease, disorder and/or a phenotypic change. Such aberrations and/or abnormalities can be rare or present at a low frequency (e.g., a few percent of the population (e.g., polymorphic)).

Disorders associated with one or more chromosome abnormalities include, but are not limited to: autosomal abnormalities (e.g., trisomies (Down syndrome (chromosome 21), Edwards syndrome (chromosome 18), Patau syndrome (chromosome 13), trisomy 9, Warkany syndrome (chromosome 8), trisomy 22/cat eye syndrome, trisomy 16); monosomies and/or deletions (Wolf-Hirschhorn syndrome (chromosome 4), Cri du chat/Chromosome 5q deletion syndrome (chromosome 5), Williams syndrome (chromosome 7), Jacobsen syndrome (chromosome 11), Miller-Dieker syndrome/Smith-Magenis syndrome (chromosome 17), Di George's syndrome (chromosome 22), genomic imprinting (Angelman syndrome/Prader-Willi syndrome (chromosome 15))); X/Y-linked abnormalities (e.g., monosomies (Turner syndrome (XO), trisomy or tetrasomy and/or other karyotypes or mosaics (Klinefelter's syndrome (47 (XXY)), 48 (XXYY), 48 (XXXY), 49 (XXXYY), 49 (XXXXY), Triple X syndrome (47 (XXX)), 48 (XXXX), 49 (XXXXX), 47 (XYY), 48 (XYYY), 49 (XYYYY), 46 (XX/XY)); translocations (e.g., leukemia or lymphoma (e.g., lymphoid (e.g., Burkitt's lymphoma t(8 MYC; 14 IGH), follicular lymphoma t(14 IGH; 18 BCL2), mantle cell lymphoma/multiple myeloma t(11 CCND1; 14 IGH), anaplastic large cell lymphoma t(2 ALK; 5 NPM1), acute lymphoblastic leukemia) or myeloid (e.g., Philadelphia chromosome t(9 ABL; 22 BCR), acute myeloblastic leukemia with maturation t(8 RUNX1T1;21 RUNX1), acute promyelocytic leukemia t(15 PML,17 RARA), acute megakaryoblastic leukemia t(1 RBM15;22 MKL1))) or other (e.g., Ewing's sarcoma t(11 FLI1; 22 EWS), synovial sarcoma t(x SYT;18 SSX), dermatofibrosarcoma protuberans t(17 COL1A1; 22 PDGFB), myxoid liposarcoma t(12 DDIT3; 16 FUS), desmoplastic small round cell tumor t(11 WT1; 22 EWS), alveolar rhabdomyosarcoma t(2 PAX3; 13 FOXO1) t (1 PAX7; 13 FOXO1))); gonadal dysgenesis (e.g., mixed gonadal dysgenesis, XX gonadal dysgenesis); and other abnormalities (e.g., fragile X syndrome, uniparental disomy). Disorders associated with one or more chromosome abnormalities also include, but are not limited to, Beckwith-Wiedmann syndrome, branchio-oto-renal syndrome, Cri-du-Chat syndrome, De Lange syndrome, holoprosencephaly, Rubinstein-Taybi syndrome and WAGR syndrome.

Disorders associated with one or more chromosome abnormalities also include cellular proliferative disorders (e.g., cancer). As used herein, the term “cellular proliferative disorder” includes disorders characterized by undesirable or inappropriate proliferation of one or more subset(s) of cells in a multicellular organism. The term “cancer” refers to various types of malignant neoplasms, most of which can invade surrounding tissues, and may metastasize to different sites (see, for example, PDR Medical Dictionary 1st edition, 1995). The terms “neoplasm” and “tumor” refer to an abnormal tissue that grows by cellular proliferation more rapidly than normal and continues to grow after the stimuli that initiated proliferation is removed (see, for example, PDR Medical Dictionary 1st edition, 1995). Such abnormal tissue shows partial or complete lack of structural organization and functional coordination with the normal tissue which may be either benign (i.e., benign tumor) or malignant (i.e., malignant tumor).

Disorders associated with one or more chromosome abnormalities also include brain disorders including, but not limited to, acoustic neuroma, acquired brain injury, Alzheimer's disease, amyotrophic lateral diseases, aneurism, aphasia, arteriovenous malformation, attention deficit hyperactivity disorder, autism Batten disease, Bechet's disease, blepharospasm, brain tumor, cerebral palsy Charcot-Marie-Tooth disease, chiari malformation, CIDP, non-Alzheimer-type dementia, dysautonomia, dyslexia, dysprazia, dystonia, epilepsy, essential tremor, Friedrich's ataxia, gaucher disease, Gullian-Barre syndrome, headache, migraine, Huntington's disease, hydrocephalus, Meniere's disease, motor neuron disease, multiple sclerosis, muscular dystrophy, myasthenia gravis, narcolepsy, Parkinson's disease, peripheral neuropathy, progressive supranuclear palsy, restless legs syndrome, Rett syndrome, schizophrenia, Shy Drager syndrome, stroke, subarachnoid hemorrhage, Sydenham's syndrome, Tay-Sachs disease, Tourett syndrome, transient ischemic attack, transverse myelitis, trigeminal neuralgia, tuberous sclerosis and von Hippel-Lindau syndrome.

As used herein, the term “retrievable label” refers to a label that is attached to a polynucleotide (e.g., a probe and/or an Oligopaint) and can, optionally, be used to specifically and/or nonspecifically bind a target protein, peptide, DNA sequence, RNA sequence, carbohydrate or the like at or near the nucleotide sequence to which one or more probes and/or Oligopaints have hybridized. In certain aspects, target proteins include, but are not limited to, proteins that are involved with gene regulation such as, e.g., proteins associated with chromatin (See, e.g., Dejardin and Kingston (2009) Cell 136:175), proteins that regulate (upregulate or downregulate) methylation, proteins that regulate (upregulate or downregulate) histone acetylation, proteins that regulate (upregulate or downregulate) transcription, proteins that regulate (upregulate or downregulate) post-transcriptional regulation, proteins that regulate (upregulate or downregulate) RNA transport, proteins that regulate (upregulate or downregulate) mRNA degradation, proteins that regulate (upregulate or downregulate) translation, proteins that regulate (upregulate or downregulate) post-translational modifications and the like.

In certain aspects, a retrievable label is activatable. As used herein, the term “activatable” refers to a retrievable label that is inert (i.e., does not bind a target) until activated (e.g., by exposure of the activatable, retrievable label to light, heat, one or more chemical compounds or the like). In other aspects, a retrievable label can bind one or more targets without the need for activation of the retrievable label.

In certain exemplary embodiments, a polynucleotide (e.g., a probe and/or an Oligopaint) has a detectable label bound thereto. As used herein, the term “detectable label” refers to a label that is attached to a polynucleotide (e.g., a probe and/or an Oligopaint) and can be used to identify a target (e.g., a chromosome or a sub-chromosomal region) to which one or more Oligopaints have hybridized. Typically, a detectable label is attached to the 3′- or 5′-end of a polynucleotide (e.g., a probe and/or an Oligopaint). Alternatively, a detectable label is attached to an internal portion of an oligonucleotide (i.e., not at the 3′ or the 5′ end). Detectable labels may vary widely in size and compositions; the following references provide guidance for selecting oligonucleotide tags appropriate for particular embodiments: Brenner, U.S. Pat. No. 5,635,400; Brenner et al., Proc. Natl. Acad. Sci., 97: 1665; Shoemaker et al. (1996) Nature Genetics, 14:450; Morris et al., EP Patent Pub. 0799897A1; Wallace, U.S. Pat. No. 5,981,179; and the like. In certain exemplary embodiments, a polynucleotide (e.g., an Oligopaint) including one or more detectable labels can have a length within a range of from 4 to 36 nucleotides, or from 6 to 30 nucleotides, or from 8 to 20 nucleotides, respectively. In other exemplary embodiments a polynucleotide (e.g., a probe and/or an Oligopaint) including one or more detectable labels can have a length of at least 30 nucleotides, at least 40 nucleotides, at least 50 nucleotides, at least 60 nucleotides, at least 70 nucleotides, at least 80 nucleotides, at least 90 nucleotides, at least 100 nucleotides, at least 150 nucleotides, at least 200 nucleotides, at least 300 nucleotides, at least 400 nucleotides, at least 500 nucleotides, at least 600 nucleotides, at least 700 nucleotides, at least 800 nucleotides, at least 900 nucleotides, at least 1000 nucleotides or greater.

Methods for incorporating detectable labels into nucleic acid probes are well known. Typically, detectable labels (e.g., as hapten- or fluorochrome-conjugated deoxyribonucleotides) are incorporated into an oligopaint during a polymerization or amplification step, e.g., by PCR, nick translation, random primer labeling, terminal transferase tailing (e.g., one or more labels can be added after cleavage of the primer sequence), and others (see Ausubel et al., 1997, Current Protocols In Molecular Biology, Greene Publishing and Wiley-Interscience, New York).

In certain aspects, a suitable retrievable label or detectable label includes, but is not limited to, a capture moiety such as a hydrophobic compound, an oligonucleotide, an antibody or fragment of an antibody, a protein, a peptide, a chemical cross-linker, an intercalator, a molecular cage (e.g., within a cage or other structure, e.g., protein cages, fullerene cages, zeolite cages, photon cages, and the like), or one or more elements of a capture pair, e.g., biotin-avidin, biotin-streptavidin, NHS-ester and the like, a thioether linkage, static charge interactions, van der Waals forces and the like (See, e.g., Holtke et al., U.S. Pat. Nos. 5,344,757; 5,702,888; and U.S. Pat. No. 5,354,657; Huber et al., U.S. Pat. No. 5,198,537; Miyoshi, U.S. Pat. No. 4,849,336; Misiura and Gait, PCT publication WO 91/17160). In certain aspects, a suitable retrievable label or detectable label is an enzyme (e.g., a methylase and/or a cleaving enzyme). In one aspect, an antibody specific against the enzyme can be used to retrieve or detect the enzyme and accordingly, retrieve or detect an oligonucleotide sequence attached to the enzyme. In another aspect, an antibody specific against the enzyme can be used to retrieve or detect the enzyme and, after stringent washes, retrieve or detect an first oligonucleotide sequence that is hybridized to a second oligonucleotide sequence having the enzyme attached thereto.

Biotin, or a derivative thereof, may be used as an oligonucleotide (e.g., a probe and/or an Oligopaint) label (e.g., as a retrievable label and/or a detectable label), and subsequently bound by a avidin/streptavidin derivative (e.g., detectably labeled, e.g., phycoerythrin-conjugated streptavidin), or an anti-biotin antibody (e.g., a detectably labeled antibody). Digoxigenin may be incorporated as a label and subsequently bound by a detectably labeled anti-digoxigenin antibody (e.g., a detectably labeled antibody, e.g., fluoresceinated anti-digoxigenin). An aminoallyl-dUTP residue may be incorporated into an oligonucleotide and subsequently coupled to an N-hydroxy succinimide (NHS) derivatized fluorescent dye, such as those listed infra. In general, any member of a conjugate pair may be incorporated into a retrievable label and/or a detectable label provided that a detectably labeled conjugate partner can be bound to permit detection. As used herein, the term antibody refers to an antibody molecule of any class, or any sub-fragment thereof, such as an Fab.

Other suitable labels (retrievable labels and/or detectable labels) include, but are not limited to, fluorescein (FAM), digoxigenin, dinitrophenol (DNP), dansyl, biotin, bromodeoxyuridine (BrdU), hexahistidine (6×His), phosphor-amino acids (e.g. P-tyr, P-ser, P-thr) and the like. In one embodiment the following hapten/antibody pairs are used for retrieval and/or detection: biotin/α-biotin, digoxigenin/α-digoxigenin, dinitrophenol (DNP)/α-DNP, 5-Carboxyfluorescein (FAM)/α-FAM.

Additional suitable labels (retrievable labels and/or detectable labels) include, but are not limited to, chemical cross-linking agents. Cross-linking agents typically contain at least two reactive groups that are reactive towards numerous groups, including, but not limited to, sulfhydryls and amines, and create chemical covalent bonds between two or more molecules. Functional groups that can be targeted with cross-linking agents include, but are not limited to, primary amines, carboxyls, sulfhydryls, carbohydrates and carboxylic acids. Protein molecules have many of these functional groups and therefore proteins and peptides can be readily conjugated using cross-linking agents. Cross-linking agents are well known in the art and are commercially available (Thermo Scientific (Rockford, Ill.)).

Fluorescent labels and their attachment to oligonucleotides (e.g., to Oligopaints) are described in many reviews, including Haugland, Handbook of Fluorescent Probes and Research Chemicals, Ninth Edition (Molecular Probes, Inc., Eugene, 2002); Keller and Manak, DNA Probes, 2nd Edition (Stockton Press, New York, 1993); Eckstein, editor, Oligonucleotides and Analogues: A Practical Approach (IRL Press, Oxford, 1991); Wetmur, Critical Reviews in Biochemistry and Molecular Biology, 26:227-259 (1991); and the like. In certain aspects, a fluorescent label is added to an oligonucleotide using terminal transferase. Particular methodologies applicable to the Oligopaint methods and compositions described herein are disclosed in the following sample of references: Fung et al., U.S. Pat. No. 4,757,141; Hobbs, Jr., et al. U.S. Pat. No. 5,151,507; Cruickshank, U.S. Pat. No. 5,091,519. In one embodiment, one or more fluorescent dyes are used as labels for Oligopaints, e.g., as disclosed by Menchen et al., U.S. Pat. No. 5,188,934 (4,7-dichlorofluorscein dyes); Begot et al., U.S. Pat. No. 5,366,860 (spectrally resolvable rhodamine dyes); Lee et al., U.S. Pat. No. 5,847,162 (4,7-dichlororhodamine dyes); Khanna et al., U.S. Pat. No. 4,318,846 (ether-substituted fluorescein dyes); Lee et al., U.S. Pat. No. 5,800,996 (energy transfer dyes); Lee et al., U.S. Pat. No. 5,066,580 (xanthine dyes): Mathies et al., U.S. Pat. No. 5,688,648 (energy transfer dyes); and the like. Labelling can also be carried out with quantum dots, as disclosed in the following patents and patent publications: U.S. Pat. Nos. 6,322,901; 6,576,291; 6,423,551; 6,251,303; 6,319,426; 6,426,513; 6,444,143; 5,990,479; 6,207,392; 2002/0045045; 2003/0017264; and the like. Amines can be incorporated into Oligopaints, and labels can be added via the amines using methods known in the art. As used herein, the term “fluorescent label” includes a signaling moiety that conveys information through the fluorescent absorption and/or emission properties of one or more molecules. Such fluorescent properties include fluorescence intensity, fluorescence life time, emission spectrum characteristics, energy transfer and the like.

Commercially available fluorescent nucleotide analogues readily incorporated into the Oligopaints include, for example, Cy3-dCTP, Cy3-dUTP, Cy5-dCTP, Cy5-dUTP (Amersham Biosciences, Piscataway, N.J.), fluorescein-12-dUTP, tetramethylrhodamine-6-dUTP, TEXAS RED™-5-dUTP, CASCADE BLUE™-7-dUTP, BODIPY TMFL-14-dUTP, BODIPY TMR-14-dUTP, BODIPY TMTR-14-dUTP, RHODAMINE GREEN™-5-dUTP, OREGON GREENR™ 488-5-dUTP, TEXAS RED™-12-dUTP, BODIPY™ 630/650-14-dUTP, BODIPY™ 650/665-14-dUTP, ALEXA FLUOR™ 488-5-dUTP, ALEXA FLUOR™ 532-5-dUTP, ALEXA FLUOR™ 568-5-dUTP, ALEXA FLUOR™ 594-5-dUTP, ALEXA FLUOR™ 546-14-dUTP, fluorescein-12-UTP, tetramethylrhodamine-6-UTP, TEXAS RED™-5-UTP, mCherry, CASCADE BLUE™-7-UTP, BODIPY™ FL-14-UTP, BODIPY TMR-14-UTP, BODIPY™ TR-14-UTP, RHODAMINE GREEN™-5-UTP, ALEXA FLUOR™ 488-5-UTP, ALEXA FLUOR™ 546-14-UTP (Molecular Probes, Inc. Eugene, Oreg.). Protocols are available for custom synthesis of nucleotides having other fluorophores. Henegariu et al., “Custom Fluorescent-Nucleotide Synthesis as an Alternative Method for Nucleic Acid Labeling,” Nature Biotechnol. 18:345-348 (2000).

Other fluorophores available for post-synthetic attachment include, inter alia, ALEXA FLUOR™ 350, ALEXA FLUOR™ 532, ALEXA FLUOR™ 546, ALEXA FLUOR™ 568, ALEXA FLUOR™ 594, ALEXA FLUOR™ 647, BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethyl rhodamine, DYLIGHT™ DYES (e.g., DYLIGHT™ 405, DYLIGHT™ 488, DYLIGHT™ 549, DYLIGHT™ 594, DYLIGHT™ 633, DYLIGHT™ 649, DYLIGHT™ 680, DYLIGHT™ 750, DYLIGHT™ 800 and the like) (available from Thermo Fisher Scientific, Rockford, Ill.), Texas Red (available from Molecular Probes, Inc., Eugene, Oreg.), and Cy2, Cy3.5, Cy5.5, and Cy7 (available from Amersham Biosciences, Piscataway, N.J. USA, and others).

FRET tandem fluorophores may also be used, such as PerCP-Cy5.5, PE-Cy5, PE-Cy5.5, PE-Cy7, PE-Texas Red, and APC-Cy7; also, PE-Alexa dyes (610, 647, 680) and APC-Alexa dyes.

Metallic silver particles may be coated onto the surface of the array to enhance signal from fluorescently labeled oligonucleotide sequences bound to an array. Lakowicz et al. (2003) BioTechniques 34:62.

Detection method(s) used will depend on the particular detectable labels used in the Oligopaints. In certain exemplary embodiments, chromosomes and/or chromosomal regions having one or more Oligopaints bound thereto may be selected for and/or screened for using a microscope, a spectrophotometer, a tube luminometer or plate luminometer, x-ray film, a scintillator, a fluorescence activated cell sorting (FACS) apparatus, a microfluidics apparatus or the like.

When fluorescently labeled Oligopaints are used, fluorescence photomicroscopy can be used to detect and record the results of in situ hybridization using routine methods known in the art. Alternatively, digital (computer implemented) fluorescence microscopy with image-processing capability may be used. Two well-known systems for imaging FISH of chromosomes having multiple colored labels bound thereto include multiplex-FISH (M-FISH) and spectral karyotyping (SKY). See Schrock et al. (1996) Science 273:494; Roberts et al. (1999) Genes Chrom. Cancer 25:241; Fransz et al. (2002) Proc. Natl. Acad. Sci. USA 99:14584; Bayani et al. (2004) Curr. Protocol. Cell Biol. 22.5.1-22.5.25; Danilova et al. (2008) Chromosoma 117:345; U.S. Pat. No. 6,066,459; and FISH TAG™ DNA Multicolor Kit instructions (Molecular probes) for a review of methods for painting chromosomes and detecting painted chromosomes.

In certain exemplary embodiments, images of fluorescently labeled chromosomes are detected and recorded using a computerized imaging system such as the Applied Imaging Corporation CytoVision System (Applied Imaging Corporation, Santa Clara, Calif.) with modifications (e.g., software, Chroma 84000 filter set, and an enhanced filter wheel). Other suitable systems include a computerized imaging system using a cooled CCD camera (Photometrics, NU200 series equipped with Kodak KAF 1400 CCD) coupled to a Zeiss Axiophot microscope, with images processed as described by Ried et al. (1992) Proc. Natl. Acad. Sci. USA 89:1388). Other suitable imaging and analysis systems are described by Schrock et al., supra; and Speicher et al., supra.

The methods and compositions described herein can be performed using a variety of biological or clinical samples, in cells that are in any (or all) stage(s) of the cell cycle (e.g., mitosis, meiosis, interphase, G0, G1, S and/or G2). As used herein, the term “sample” include all types of cell culture, animal or plant tissue, peripheral blood lymphocytes, buccal smears, touch preparations prepared from uncultured primary tumors, cancer cells, bone marrow, cells obtained from biopsy or cells in bodily fluids (e.g., blood, urine, sputum and the like), cells from amniotic fluid, cells from maternal blood (e.g., fetal cells), cells from testis and ovary, and the like. Samples include chromosomes or portions thereof, nucleic acids (e.g., polynucleotides, oligonucleotides and the like), amino acids (e.g., proteins, protein fragments polypeptides, peptides, etc.), antibodies, small molecules, pharmaceuticals, biologics and the like. Samples are prepared for assays of the invention using conventional techniques, which typically depend on the source from which a sample or specimen is taken. These examples are not to be construed as limiting the sample types applicable to the methods and/or compositions described herein.

In certain exemplary embodiments, probes and/or Oligopaints include multiple chromosome-specific probes, which are differentially labeled (i.e., at least two of the chromosome-specific probes are differently labeled). Various approaches to multi-color chromosome painting have been described in the art and can be adapted to the present invention following the guidance provided herein. Examples of such differential labeling (“multicolor FISH”) include those described by Schrock et al. (1996) Science 273:494, and Speicher et al. (1996) Nature Genet. 12:368). Schrock et al. describes a spectral imaging method, in which epifluorescence filter sets and computer software is used to detect and discriminate between multiple differently labeled DNA probes hybridized simultaneously to a target chromosome set. Speicher et al. describes using different combinations of 5 fluorochromes to label each of the human chromosomes (or chromosome arms) in a 27-color FISH termed “combinatorial multifluor FISH”). Other suitable methods may also be used (see, e.g., Ried et al., 1992, Proc. Natl. Acad. Sci. USA 89:1388-92).

Hybridization of probes and/or Oligopaints to target chromosomes sequences can be accomplished by standard in situ hybridization (ISH) techniques (see, e.g., Gall and Pardue (1981) Meth. Enzymol. 21:470; Henderson (1982) Int. Review of Cytology 76:1), as well as by any of the FISH methods described further herein. Generally, ISH comprises the following major steps: (1) fixation of the biological structure to be analyzed (e.g., a chromosome spread), (2) pre-hybridization treatment of the biological structure to increase accessibility of target DNA (e.g., denaturation with heat or alkali), (3) optional pre-hybridization treatment to reduce nonspecific binding (e.g., by blocking the hybridization capacity of repetitive sequences), (4) hybridization of the mixture of nucleic acids to the nucleic acid in the biological structure or tissue; (5) post-hybridization washes to remove nucleic acid fragments not bound in the hybridization and (6) detection of the hybridized labelled oligonucleotides (e.g., hybridized Oligopaints). The reagents used in each of these steps and their conditions of use vary depending on the particular situation. For instance, step 3 will not always be necessary as the Oligopaints described herein can be designed to avoid repetitive sequences). Hybridization conditions are also described in U.S. Pat. No. 5,447,841. It will be appreciated that numerous variations of in situ hybridization protocols and conditions are known and may be used in conjunction with the present invention by practitioners following the guidance provided herein.

As used herein, the term “hybridization” refers to the process in which two single-stranded polynucleotides bind non-covalently to form a stable double-stranded polynucleotide. The term “hybridization” may also refer to triple-stranded hybridization. The resulting (usually) double-stranded polynucleotide is a “hybrid” or “duplex.” “Hybridization conditions” will typically include salt concentrations of less than about 1 M, more usually less than about 500 mM and even more usually less than about 200 mM. Hybridization temperatures can be as low as 5° C. or lower, but are typically greater than 22° C., more typically greater than about 30° C., and often in excess of about 37° C. Hybridizations are usually performed under stringent conditions, i.e., conditions under which a probe will hybridize to its target subsequence. Stringent conditions are sequence-dependent and are different in different circumstances. Longer fragments may require higher hybridization temperatures for specific hybridization. As other factors may affect the stringency of hybridization, including base composition and length of the complementary strands, presence of organic solvents and extent of base mismatching, the combination of parameters is more important than the absolute measure of any one alone. Generally, stringent conditions are selected to be about 5° C. lower than the Tₘ for the specific sequence at s defined ionic strength and pH. Exemplary stringent conditions include salt concentration of at least 0.01 M to no more than 1 M Na ion concentration (or other salts) at a pH 7.0 to 8.3 and a temperature of at least 25° C. For example, conditions of 5×SSPE (750 mM NaCl, 50 mM Na phosphate, 5 mM EDTA, pH 7.4) and a temperature of 25-30° C. are suitable for allele-specific probe hybridizations. For stringent conditions, see for example, Sambrook, Fritsche and Maniatis, Molecular Cloning A Laboratory Manual, 2nd Ed. Cold Spring Harbor Press (1989) and Anderson Nucleic Acid Hybridization, 1^{st} Ed., BIOS Scientific Publishers Limited (1999). “Hybridizing specifically to” or “specifically hybridizing to” or like expressions refer to the binding, duplexing, or hybridizing of a molecule substantially to or only to a particular nucleotide sequence or sequences under stringent conditions when that sequence is present in a complex mixture (e.g., total cellular) DNA or RNA.

In certain exemplary embodiments, probes and/or Oligopaints are complementary to genomic nucleic sequences that are present in low or single copy numbers (e.g., genomic nucleic sequences that are not repetitive elements). As used herein, the term “repetitive element” refers to a DNA sequence that is present in many identical or similar copies in the genome. Repetitive elements are not intended to refer to a DNA sequence that is present on each copy of the same chromosome (e.g., a DNA sequence that is present only once, but is found on both copies of chromosome 11, would not be considered a repetitive element, and would be considered a sequence that is present in the genome as one copy). The genome consists of three broad sequence components: Single copy or at least very low copy number DNA (approximately 60% of the human genome); moderately repetitive elements (approximately 30% of the human genome); and highly repetitive elements (approximately 10% of the human genome). For a review, see Human Molecular Genetics, Chapter 7 (1999), John Wiley & Sons, Inc.

In certain exemplary embodiments, methods and compositions described herein include the use of a support, e.g., a separable multi-well apparatus. In certain aspects, multiple supports (tens, hundreds, thousands or more) may be utilized (e.g., synthesized, amplified, hybridized or the like) in parallel. Suitable supports include, but are not limited to, slides (e.g., microscope slides), beads, chips, particles, strands, gels, sheets, tubing (e.g., microfuge tubes, test tubes, cuvettes), spheres, containers, capillaries, microfibers, pads, slices, films, plates (e.g., multi-well plates (e.g., a separable multi-well apparatus)), microfluidic supports (e.g., microarray chips, flow channel plates, biochips and the like) and the like. In various embodiments, the solid supports may be biological, nonbiological, organic, inorganic or combinations thereof. When using supports that are substantially planar, the support may be physically separated into regions, for example, with trenches, grooves, wells, or chemical barriers (e.g., lacking a lipid-binding coating).

In certain exemplary embodiments, supports may have functional groups on their surface which can be used to attach a lipid bilayer (e.g., a phospholipid bilayer) to the support. For example, at least a portion of the support can be coated with silane and dextran (e.g., high molecular weight dextran). Dextran in its hydrated form can function as a molecular cushion for the membrane and is capable of binding lipids on the support. Suitable functional groups include, but are not limited to, silicon oxides (e.g., SiO₂), MgF₂, CaF₂, mica, polyacrylamide, dextran and the like and any combination thereof.

In certain exemplary embodiments, methods of generating and amplifying synthetic oligonucleotide sequences, e.g., Oligopaint sequences, are provided. As used herein, the term “oligonucleotide” is intended to include, but is not limited to, a single-stranded DNA or RNA molecule, typically prepared by synthetic means. Nucleotides of the present invention will typically be the naturally-occurring nucleotides such as nucleotides derived from adenosine, guanosine, uridine, cytidine and thymidine. When oligonucleotides are referred to as “double-stranded,” it is understood by those of skill in the art that a pair of oligonucleotides exists in a hydrogen-bonded, helical array typically associated with, for example, DNA. In addition to the 100% complementary form of double-stranded oligonucleotides, the term “double-stranded” as used herein is also meant to include those form which include such structural features as bulges and loops (see Stryer, Biochemistry, Third Ed. (1988), incorporated herein by reference in its entirety for all purposes). As used herein, the term “polynucleotide” is intended to include, but is not limited to, two or more oligonucleotides joined together (e.g., by hybridization, ligation, polymerization and the like).

The term “operably linked,” when describing the relationship between two nucleic acid regions, refers to a juxtaposition wherein the regions are in a relationship permitting them to function in their intended manner. For example, a control sequence “operably linked” to a coding sequence is ligated in such a way that expression of the coding sequence is achieved under conditions compatible with the control sequences, such as when the appropriate molecules (e.g., inducers and polymerases) are bound to the control or regulatory sequence(s).

In certain exemplary embodiments, nucleotide analogs or derivatives will be used, such as nucleosides or nucleotides having protecting groups on either the base portion or sugar portion of the molecule, or having attached or incorporated labels, or isosteric replacements which result in monomers that behave in either a synthetic or physiological environment in a manner similar to the parent monomer. The nucleotides can have a protecting group which is linked to, and masks, a reactive group on the nucleotide. A variety of protecting groups are useful in the invention and can be selected depending on the synthesis techniques employed and are discussed further below. After the nucleotide is attached to the support or growing nucleic acid, the protecting group can be removed.

Oligonucleotides or fragments thereof may be purchased from commercial sources. Oligonucleotide sequences may be prepared by any suitable method, e.g., the phosphoramidite method described by Beaucage and Carruthers ((1981) Tetrahedron Lett. 22: 1859) or the triester method according to Matteucci et al. (1981) J. Am. Chem. Soc. 103:3185), both incorporated herein by reference in their entirety for all purposes, or by other chemical methods using either a commercial automated oligonucleotide synthesizer or high-throughput, high-density array methods described herein and known in the art (see U.S. Pat. Nos. 5,602,244, 5,574,146, 5,554,744, 5,428,148, 5,264,566, 5,141,813, 5,959,463, 4,861,571 and 4,659,774, incorporated herein by reference in its entirety for all purposes). Pre-synthesized oligonucleotides and chips containing oligonucleotides may also be obtained commercially from a variety of vendors.

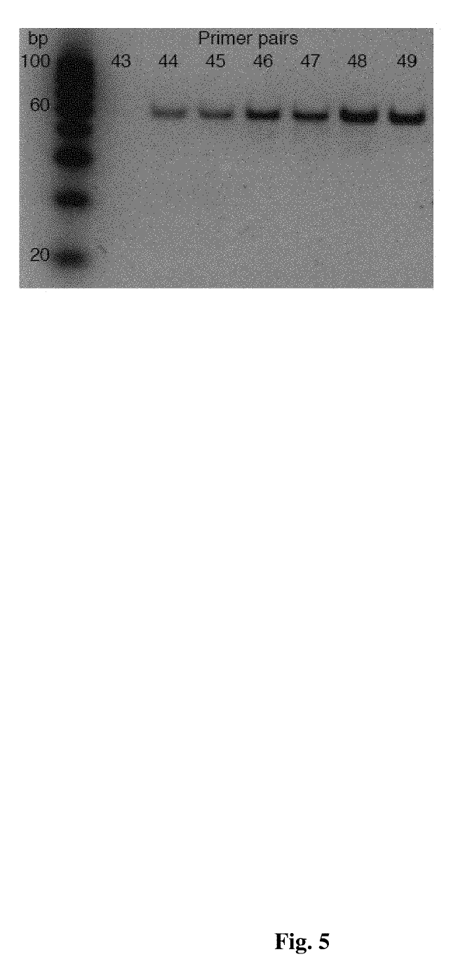

In an exemplary embodiment, oligonucleotides may be synthesized on a solid support using maskless array synthesizer (MAS). Maskless array synthesizers are described, for example, in PCT application No. WO 99/42813 and in corresponding U.S. Pat. No. 6,375,903. Other examples are known of maskless instruments which can fabricate a custom DNA microarray in which each of the features in the array has a single stranded DNA molecule of desired sequence. An exemplary type of instrument is the type shown in FIG. 5 of U.S. Pat. No. 6,375,903, based on the use of reflective optics. It is a desirable that this type of maskless array synthesizer is under software control. Since the entire process of microarray synthesis can be accomplished in only a few hours, and since suitable software permits the desired DNA sequences to be altered at will, this class of device makes it possible to fabricate microarrays including DNA segments of different sequence every day or even multiple times per day on one instrument. The differences in DNA sequence of the DNA segments in the microarray can also be slight or dramatic, it makes no difference to the process. The MAS instrument may be used in the form it would normally be used to make microarrays for hybridization experiments, but it may also be adapted to have features specifically adapted for the compositions, methods, and systems described herein. For example, it may be desirable to substitute a coherent light source, i.e., a laser, for the light source shown in FIG. 5 of the above-mentioned U.S. Pat. No. 6,375,903. If a laser is used as the light source, a beam expanded and scatter plate may be used after the laser to transform the narrow light beam from the laser into a broader light source to illuminate the micromirror arrays used in the maskless array synthesizer. It is also envisioned that changes may be made to the flow cell in which the microarray is synthesized. In particular, it is envisioned that the flow cell can be compartmentalized, with linear rows of array elements being in fluid communication with each other by a common fluid channel, but each channel being separated from adjacent channels associated with neighboring rows of array elements. During microarray synthesis, the channels all receive the same fluids at the same time. After the DNA segments are separated from the substrate, the channels serve to permit the DNA segments from the row of array elements to congregate with each other and begin to self-assemble by hybridization.

Other methods for synthesizing oligonucleotides (e.g., probes and/or Oligopaints) include, for example, light-directed methods utilizing masks, flow channel methods, spotting methods, pin-based methods, and methods utilizing multiple supports.

Light directed methods utilizing masks (e.g., VLSIPS™ methods) for the synthesis of oligonucleotides is described, for example, in U.S. Pat. Nos. 5,143,854, 5,510,270 and 5,527,681. These methods involve activating predefined regions of a solid support and then contacting the support with a preselected monomer solution. Selected regions can be activated by irradiation with a light source through a mask much in the manner of photolithography techniques used in integrated circuit fabrication. Other regions of the support remain inactive because illumination is blocked by the mask and they remain chemically protected. Thus, a light pattern defines which regions of the support react with a given monomer. By repeatedly activating different sets of predefined regions and contacting different monomer solutions with the support, a diverse array of polymers is produced on the support. Other steps, such as washing unreacted monomer solution from the support, can be used as necessary. Other applicable methods include mechanical techniques such as those described in U.S. Pat. No. 5,384,261.

Additional methods applicable to synthesis and/or amplification of oligonucleotides (e.g., probes and/or Oligopaints) on a single support are described, for example, in U.S. Pat. No. 5,384,261. For example reagents may be delivered to the support by either (1) flowing within a channel defined on predefined regions or (2) “spotting” on predefined regions. Other approaches, as well as combinations of spotting and flowing, may be employed as well. In each instance, certain activated regions of the support are mechanically separated from other regions when the monomer solutions are delivered to the various reaction sites.

Flow channel methods involve, for example, microfluidic systems to control synthesis of oligonucleotides on a solid support. For example, diverse polymer sequences may be synthesized at selected regions of a solid support by forming flow channels on a surface of the support through which appropriate reagents flow or in which appropriate reagents are placed. One of skill in the art will recognize that there are alternative methods of forming channels or otherwise protecting a portion of the surface of the support. For example, a protective coating such as a hydrophilic or hydrophobic coating (depending upon the nature of the solvent) is utilized over portions of the support to be protected, sometimes in combination with materials that facilitate wetting by the reactant solution in other regions. In this manner, the flowing solutions are further prevented from passing outside of their designated flow paths.

Spotting methods for preparation of oligonucleotides on a solid support involve delivering reactants in relatively small quantities by directly depositing them in selected regions. In some steps, the entire support surface can be sprayed or otherwise coated with a solution, if it is more efficient to do so. Precisely measured aliquots of monomer solutions may be deposited dropwise by a dispenser that moves from region to region. Typical dispensers include a micropipette to deliver the monomer solution to the support and a robotic system to control the position of the micropipette with respect to the support, or an ink-jet printer. In other embodiments, the dispenser includes a series of tubes, a manifold, an array of pipettes, or the like so that various reagents can be delivered to the reaction regions simultaneously.

Pin-based methods for synthesis of oligonucleotides on a solid support are described, for example, in U.S. Pat. No. 5,288,514. Pin-based methods utilize a support having a plurality of pins or other extensions. The pins are each inserted simultaneously into individual reagent containers in a tray. An array of 96 pins is commonly utilized with a 96-container tray, such as a 96-well microtitre dish. Each tray is filled with a particular reagent for coupling in a particular chemical reaction on an individual pin. Accordingly, the trays will often contain different reagents. Since the chemical reactions have been optimized such that each of the reactions can be performed under a relatively similar set of reaction conditions, it becomes possible to conduct multiple chemical coupling steps simultaneously.

In yet another embodiment, a plurality of oligonucleotides (e.g., probes and/or Oligopaints) may be synthesized on multiple supports. One example is a bead based synthesis method which is described, for example, in U.S. Pat. Nos. 5,770,358, 5,639,603, and 5,541,061. For the synthesis of molecules such as oligonucleotides on beads, a large plurality of beads are suspended in a suitable carrier (such as water) in a container. The beads are provided with optional spacer molecules having an active site to which is complexed, optionally, a protecting group. At each step of the synthesis, the beads are divided for coupling into a plurality of containers. After the nascent oligonucleotide chains are deprotected, a different monomer solution is added to each container, so that on all beads in a given container, the same nucleotide addition reaction occurs. The beads are then washed of excess reagents, pooled in a single container, mixed and re-distributed into another plurality of containers in preparation for the next round of synthesis. It should be noted that by virtue of the large number of beads utilized at the outset, there will similarly be a large number of beads randomly dispersed in the container, each having a unique oligonucleotide sequence synthesized on a surface thereof after numerous rounds of randomized addition of bases. An individual bead may be tagged with a sequence which is unique to the double-stranded oligonucleotide thereon, to allow for identification during use.

In certain embodiments, a plurality of oligonucleotides (e.g., probes and/or Oligopaints) may be synthesized, amplified and/or used in conjunction with beads and/or bead-based arrays. As used herein, the term “bead” refers to a discrete particle that may be spherical (e.g., microspheres) or have an irregular shape. Beads may be as small as approximately 0.1 μm in diameter or as large approximately several millimeters in diameter. Beads typically range in size from approximately 0.1 μm to 200 μm in diameter. Beads may comprise a variety of materials including, but not limited to, paramagnetic materials, ceramic, plastic, glass, polystyrene, methylstyrene, acrylic polymers, titanium, latex, sepharose, cellulose, nylon and the like.

In certain aspects, beads may have functional groups on their surface which can be used to oligonucleotides (e.g., probes and/or Oligopaints) to the bead. Probe and/or Oligonucleotide sequences can be attached to a bead by hybridization (e.g., binding to a polymer), covalent attachment, magnetic attachment, affinity attachment and the like. For example, the bead can be coated with streptavidin and the nucleic acid sequence can include a biotin moiety. The biotin is capable of binding streptavidin on the bead, thus attaching the nucleic acid sequence to the bead. Beads coated with streptavidin, oligo-dT, and histidine tag binding substrate are commercially available (Dynal Biotech, Brown Deer, Wis.). Beads may also be functionalized using, for example, solid-phase chemistries known in the art, such as those for generating nucleic acid arrays, such as carboxyl, amino, and hydroxyl groups, or functionalized silicon compounds (see, for example, U.S. Pat. No. 5,919,523).

Various exemplary protecting groups useful for synthesis of oligonucleotides on a solid support are described in, for example, Atherton et al., 1989, Solid Phase Peptide Synthesis, IRL Press. In various embodiments, the methods described herein utilize solid supports for immobilization of nucleic acids. For example, oligonucleotides may be synthesized on one or more solid supports. Exemplary solid supports include, for example, slides, beads, chips, particles, strands, gels, sheets, tubing, spheres, containers, capillaries, pads, slices, films, or plates. In various embodiments, the solid supports may be biological, nonbiological, organic, inorganic, or combinations thereof. When using supports that are substantially planar, the support may be physically separated into regions, for example, with trenches, grooves, wells, or chemical barriers (e.g., hydrophobic coatings, etc.). Supports that are transparent to light are useful when the assay involves optical detection (see e.g., U.S. Pat. No. 5,545,531). The surface of the solid support will typically contain reactive groups, such as carboxyl, amino, and hydroxyl or may be coated with functionalized silicon compounds (see e.g., U.S. Pat. No. 5,919,523).

In one embodiment, the oligonucleotides synthesized on the solid support may be used as a template for the production of probes and/or Oligopaints. For example, the support bound oligonucleotides may be contacted with primers that hybridize to the oligonucleotides under conditions that permit chain extension of the primers. The support bound duplexes may then be denatured, pooled and subjected to further rounds of amplification to produce probes and/or Oligopaints in solution. In another embodiment, the support-bound oligonucleotides may be removed from the solid, pooled and amplified to produce probes and/or Oligopaints in solution. The oligonucleotides may be removed from the solid support, for example, by exposure to conditions such as acid, base, oxidation, reduction, heat, light, metal ion catalysis, displacement or elimination chemistry, or by enzymatic cleavage.

In one embodiment, oligonucleotides may be attached to a solid support through a cleavable linkage moiety. For example, the solid support may be functionalized to provide cleavable linkers for covalent attachment to the oligonucleotides. The linker moiety may be one, two, three, four, five, six or more atoms in length. Alternatively, the cleavable moiety may be within an oligonucleotide and may be introduced during in situ synthesis. A broad variety of cleavable moieties are available in the art of solid phase and microarray oligonucleotide synthesis (see e.g., Pon (1993) Methods Mol. Biol. 20:465; Verma et al. (1998) Ann. Rev. Biochem. 67:99; U.S. Pat. Nos. 5,739,386, 5,700,642 and 5,830,655; and U.S. Patent Publication Nos. 2003/0186226 and 2004/0106728). A suitable cleavable moiety may be selected to be compatible with the nature of the protecting group of the nucleoside bases, the choice of solid support, and/or the mode of reagent delivery, among others. In an exemplary embodiment, the oligonucleotides cleaved from the solid support contain a free 3′-OH end. Alternatively, the free 3′-OH end may also be obtained by chemical or enzymatic treatment, following the cleavage of oligonucleotides. The cleavable moiety may be removed under conditions which do not degrade the oligonucleotides. The linker may be cleaved using two approaches, either (a) simultaneously under the same conditions as the deprotection step or (b) subsequently utilizing a different condition or reagent for linker cleavage after the completion of the deprotection step.

The covalent immobilization site may either be at the 5′ end of the oligonucleotide or at the 3′ end of the oligonucleotide. In some instances, the immobilization site may be within the oligonucleotide (i.e. at a site other than the 5′ or 3′ end of the oligonucleotide). The cleavable site may be located along the oligonucleotide backbone, for example, a modified 3′-5′ internucleotide linkage in place of one of the phosphodiester groups, such as ribose, dialkoxysilane, phosphorothioate, and phosphoramidate internucleotide linkage. The cleavable oligonucleotide analogs may also include a substituent on, or replacement of, one of the bases or sugars, such as 7-deazaguanosine, 5-methylcytosine, inosine, uridine, and the like.

In one embodiment, cleavable sites contained within the modified oligonucleotide may include chemically cleavable groups, such as dialkoxysilane, 3′-(S)-phosphorothioate, 5′-(S)-phosphorothioate, 3′-(N)-phosphoramidate, 5′-(N)phosphoramidate, and ribose. Synthesis and cleavage conditions of chemically cleavable oligonucleotides are described in U.S. Pat. Nos. 5,700,642 and 5,830,655. For example, depending upon the choice of cleavable site to be introduced, either a functionalized nucleoside or a modified nucleoside dimer may be first prepared, and then selectively introduced into a growing oligonucleotide fragment during the course of oligonucleotide synthesis. Selective cleavage of the dialkoxysilane may be effected by treatment with fluoride ion. Phosphorothioate internucleotide linkage may be selectively cleaved under mild oxidative conditions. Selective cleavage of the phosphoramidate bond may be carried out under mild acid conditions, such as 80% acetic acid. Selective cleavage of ribose may be carried out by treatment with dilute ammonium hydroxide.

In another embodiment, a non-cleavable hydroxyl linker may be converted into a cleavable linker by coupling a special phosphoramidite to the hydroxyl group prior to the phosphoramidite or H-phosphonate oligonucleotide synthesis as described in U.S. Patent Application Publication No. 2003/0186226. The cleavage of the chemical phosphorylation agent at the completion of the oligonucleotide synthesis yields an oligonucleotide bearing a phosphate group at the 3′ end. The 3′-phosphate end may be converted to a 3′ hydroxyl end by a treatment with a chemical or an enzyme, such as alkaline phosphatase, which is routinely carried out by those skilled in the art.

In another embodiment, the cleavable linking moiety may be a TOPS (two oligonucleotides per synthesis) linker (see e.g., PCT publication WO 93/20092). For example, the TOPS phosphoramidite may be used to convert a non-cleavable hydroxyl group on the solid support to a cleavable linker. A preferred embodiment of TOPS reagents is the Universal TOPS™ phosphoramidite. Conditions for Universal TOPS™ phosphoramidite preparation, coupling and cleavage are detailed, for example, in Hardy et al, Nucleic Acids Research 22(15):2998-3004 (1994). The Universal TOPS™ phosphoramidite yields a cyclic 3′ phosphate that may be removed under basic conditions, such as the extended ammonia and/or ammonia/methylamine treatment, resulting in the natural 3′ hydroxy oligonucleotide.

In another embodiment, a cleavable linking moiety may be an amino linker. The resulting oligonucleotides bound to the linker via a phosphoramidite linkage may be cleaved with 80% acetic acid yielding a 3′-phosphorylated oligonucleotide.

In another embodiment, the cleavable linking moiety may be a photocleavable linker, such as an ortho-nitrobenzyl photocleavable linker. Synthesis and cleavage conditions of photolabile oligonucleotides on solid supports are described, for example, in Venkatesan et al. J. of Org. Chem. 61:525-529 (1996), Kahl et al., J. of Org. Chem. 64:507-510 (1999), Kahl et al., J. of Org. Chem. 63:4870-4871 (1998), Greenberg et al., J. of Org. Chem. 59:746-753 (1994), Holmes et al., J. of Org. Chem. 62:2370-2380 (1997), and U.S. Pat. No. 5,739,386. Ortho-nitobenzyl-based linkers, such as hydroxymethyl, hydroxyethyl, and Fmoc-aminoethyl carboxylic acid linkers, may also be obtained commercially.

In another embodiment, oligonucleotides may be removed from a solid support by an enzyme such as a nuclease. For example, oligonucleotides may be removed from a solid support upon exposure to one or more restriction endonucleases, including, for example, class IIs restriction enzymes. A restriction endonuclease recognition sequence may be incorporated into the immobilized oligonucleotides and the oligonucleotides may be contacted with one or more restriction endonucleases to remove the oligonucleotides from the support. In various embodiments, when using enzymatic cleavage to remove the oligonucleotides from the support, it may be desirable to contact the single stranded immobilized oligonucleotides with primers, polymerase and dNTPs to form immobilized duplexes. The duplexes may then be contacted with the enzyme (e.g., a restriction endonuclease) to remove the duplexes from the surface of the support. Methods for synthesizing a second strand on a support bound oligonucleotide and methods for enzymatic removal of support bound duplexes are described, for example, in U.S. Pat. No. 6,326,489. Alternatively, short oligonucleotides that are complementary to the restriction endonuclease recognition and/or cleavage site (e.g., but are not complementary to the entire support bound oligonucleotide) may be added to the support bound oligonucleotides under hybridization conditions to facilitate cleavage by a restriction endonuclease (see e.g., PCT Publication No. WO 04/024886).

In yet another embodiment, a plurality of oligonucleotides (e.g., Oligopaints) may be synthesized and/or amplified in solution. Methods of synthesizing oligonucleotide sequences are well-known in the art (See, e.g., Seliger (1993) Protocols for Oligonucleotides and Analogs: Synthesis and Properties, vol. 20, pp. 391-435, Efimov (2007) Nucleosides, Nucleotides & Nucleic Acids 26:8, McMinn et al. (1997) J. Org. Chem. 62:7074, Froehler et al. (1986) Nucleic Acids Res. 14:5399, Garegg (1986) Tet. Lett. 27:4051, Efimov (1983) Nucleic Acids Res. 11:8369, Reese (1978) Tetrahedron 34:3143).

In certain embodiments, oligonucleotides (e.g., probes and/or Oligopaints) are double stranded (ds). In certain aspects, a ds oligonucleotide may be synthesized as two single stranded oligonucleotides that are hybridized together, thus forming a ds oligonucleotide. Alternatively, a ds oligonucleotide may be synthesized in a ds form (e.g., using a single stranded (ss) oligonucleotide as a template). In other embodiments, oligonucleotides (e.g., probes and/or Oligopaints) are ss. In certain aspects, a ss oligonucleotide is generated in a ss form. In other aspects, a ss oligonucleotide is synthesized in a ds form and is converted to ss form subsequent to synthesis using any of a variety of methods well known in the art (e.g., by incorporating dUs into the ds oligonucleotide during synthesis that can be cleaved after synthesis, by chemical cleavage after synthesis, by enzymatic cleavage after synthesis, by nuclease digestion after synthesis, by light based cleavage after synthesis and the like).

Exemplary chemically cleavable internucleotide linkages for use in the methods described herein include, for example, β-cyano ether, 5′-deoxy-5′-aminocarbamate, 3′deoxy-3′-aminocarbamate, urea, 2′cyano-3′,5′-phosphodiester, 3′45)-phosphorothioate, 5′-(S)-phosphorothioate, 3′-(N)-phosphoramidate, 5′-(N)-phosphoramidate, α-amino amide, vicinal diol, ribonucleoside insertion, 2′-amino-3′,5′-phosphodiester, allylic sulfoxide, ester, silyl ether, dithioacetal, 5′-thio-furmal, α-hydroxy-methyl-phosphonic bisamide, acetal, 3′-thio-furmal, methylphosphonate and phosphotriester. Internucleoside silyl groups such as trialkylsilyl ether and dialkoxysilane are cleaved by treatment with fluoride ion. Base-cleavable sites include β-cyano ether, 5′-deoxy-5′-aminocarbamate, 3′-deoxy-3′-aminocarbamate, urea, 2′-cyano-3′, 5′-phosphodiester, 2′-amino-3′, 5′-phosphodiester, ester and ribose. Thio-containing internucleotide bonds such as 3′-(S)-phosphorothioate and 5′-(S)-phosphorothioate are cleaved by treatment with silver nitrate or mercuric chloride. Acid cleavable sites include 3′-(N)-phosphoramidate, 5′-(N)-phosphoramidate, dithioacetal, acetal and phosphonic bisamide. An α-aminoamide internucleoside bond is cleavable by treatment with isothiocyanate, and titanium may be used to cleave a 2′-amino-3′,5′-phosphodiester-O-ortho-benzyl internucleoside bond. Vicinal diol linkages are cleavable by treatment with periodate. Thermally cleavable groups include allylic sulfoxide and cyclohexene while photo-labile linkages include nitrobenzylether and thymidine dimer. Methods synthesizing and cleaving nucleic acids containing chemically cleavable, thermally cleavable, and photo-labile groups are described for example, in U.S. Pat. No. 5,700,642.

Enzymatic cleavage may be mediated by including a restriction endonuclease cleavage site in the oligonucleotide sequence. After synthesis of a ds oligonucleotide, the ds oligonucleotide may be contacted with one or more endonucleases to remove one strand. A wide variety of restriction endonucleases having specific binding and/or cleavage sites are commercially available, for example, from New England Biolabs (Ipswich, Mass.).

In various embodiments, the methods disclosed herein comprise amplification of oligonucleotide sequences including, for example, Oligopaints. Amplification methods may comprise contacting a nucleic acid with one or more primers that specifically hybridize to the nucleic acid under conditions that facilitate hybridization and chain extension. Exemplary methods for amplifying nucleic acids include the polymerase chain reaction (PCR) (see, e.g., Mullis et al. (1986) Cold Spring Harb. Symp. Quant. Biol. 51 Pt 1:263 and Cleary et al. (2004) Nature Methods 1:241; and U.S. Pat. Nos. 4,683,195 and 4,683,202), anchor PCR, RACE PCR, ligation chain reaction (LCR) (see, e.g., Landegran et al. (1988) Science 241:1077-1080; and Nakazawa et al. (1994) Proc. Natl. Acad. Sci. U.S.A. 91:360-364), self sustained sequence replication (Guatelli et al. (1990) Proc. Natl. Acad. Sci. U.S.A. 87:1874), transcriptional amplification system (Kwoh et al. (1989) Proc. Natl. Acad. Sci. U.S.A. 86:1173), Q-Beta Replicase (Lizardi et al. (1988) BioTechnology 6:1197), recursive PCR (Jaffe et al. (2000) J. Biol. Chem. 275:2619; and Williams et al. (2002) J. Biol. Chem. 277:7790), the amplification methods described in U.S. Pat. Nos. 6,391,544, 6,365,375, 6,294,323, 6,261,797, 6,124,090 and 5,612,199, or any other nucleic acid amplification method using techniques well known to those of skill in the art. In exemplary embodiments, the methods disclosed herein utilize PCR amplification.

In certain exemplary embodiments, universal primers will be used to amplify nucleic acid sequences such as, for example, probes and/or Oligopaints. The term “universal primers” refers to a set of primers (e.g., a forward and reverse primer) that may be used for chain extension/amplification of a plurality of polynucleotides, e.g., the primers hybridize to sites that are common to a plurality of polynucleotides. For example, universal primers may be used for amplification of all, or essentially all, polynucleotides in a single pool. In certain aspects, forward primers and reverse primers have the same sequence. In other aspects, the sequence of forward primers differs from the sequence of reverse primers. In still other aspects, a plurality of universal primers are provided, e.g., tens, hundreds, thousands or more.

In certain embodiments, the universal primers may be temporary primers that may be removed after amplification via enzymatic or chemical cleavage. In certain embodiments, the universal primers may be temporary primers that may be removed after amplification via enzymatic or chemical cleavage. In other embodiments, the universal primers may comprise a modification that becomes incorporated into the polynucleotide molecules upon chain extension. Exemplary modifications include, for example, a 3′ or 5′ end cap, a label (e.g., fluorescein), or a tag (e.g., a tag that facilitates immobilization or isolation of the polynucleotide, such as, biotin, etc.).

In exemplary embodiments, primers may be designed to be temporary to permit removal of the primers. Temporary primers may be designed so as to be removable by chemical, thermal, light based, or enzymatic cleavage. Cleavage may occur upon addition of an external factor (e.g., an enzyme, chemical, heat, light, etc.) or may occur automatically after a certain time period (e.g., after n rounds of amplification). In one embodiment, temporary primers may be removed by chemical cleavage. For example, primers having acid labile or base labile sites may be used for amplification. The amplified pool may then be exposed to acid or base to remove the primer at the desired location. Alternatively, the temporary primers may be removed by exposure to heat and/or light. For example, primers having heat labile or photolabile sites may be used for amplification. The amplified pool may then be exposed to heat and/or light to remove the primer/primer binding sites at the desired location. In another embodiment, an RNA primer may be used for amplification thereby forming short stretches of RNA/DNA hybrids at the ends of the nucleic acid molecule. The primer site may then be removed by exposure to an RNase (e.g., RNase H). In various embodiments, the method for removing the primer may only cleave a single strand of the amplified duplex thereby leaving 3′ or 5′ overhangs. Such overhangs may be removed using an exonuclease to form blunt ended double stranded duplexes. For example, RecJf may be used to remove single stranded 5′ overhangs and Exonuclease I or Exonuclease T may be used to remove single stranded 3′ overhangs. Additionally, Si nuclease, Pi nuclease, mung bean nuclease, and CEL I nuclease, may be used to remove single stranded regions from a nucleic acid molecule. RecJf, Exonuclease I, Exonuclease T, and mung bean nuclease are commercially available, for example, from New England Biolabs (Ipswich, Mass.). 51 nuclease, P1 nuclease and CEL I nuclease are described, for example, in Vogt, V. M., Eur. J. Biochem., 33: 192-200 (1973); Fujimoto et al., Agric. Biol. Chem. 38: 777-783 (1974); Vogt, V. M., Methods Enzymol. 65: 248-255 (1980); and Yang et al., Biochemistry 39: 3533-3541 (2000).

In one embodiment, the temporary primers may be removed from a nucleic acid by chemical, thermal, or light based cleavage as described supra. In other embodiments, primers may be removed using enzymatic cleavage. For example, primers may be designed to include a restriction endonuclease cleavage site. After amplification, the pool of nucleic acids may be contacted with one or more endonucleases to produce double stranded breaks thereby removing the primers. In certain embodiments, the forward and reverse primers may be removed by the same or different restriction endonucleases. Any type of restriction endonuclease may be used to remove the primers/primer binding sites from nucleic acid sequences. In various embodiments, restriction endonucleases that produce 3′ overhangs, 5′ overhangs or blunt ends may be used.

In certain embodiments, it may be desirable to utilize a primer comprising one or more modifications such as a cap (e.g., to prevent exonuclease cleavage), a linking moiety (such as those described above to facilitate immobilization of an oligonucleotide onto a substrate), or a label (e.g., to facilitate detection, isolation and/or immobilization of a nucleic acid construct). Suitable modifications include, for example, various enzymes, prosthetic groups, luminescent markers, bioluminescent markers, fluorescent markers (e.g., fluorescein), radiolabels (e.g., ³²P, ³⁵S, etc.), biotin, polypeptide epitopes, etc. as described further herein.

Embodiments of the present invention are directed to oligonucleotide sequences (e.g., Oligopaints) having one or more amplification sequences or amplification sites. As used herein, the term “amplification site” is intended to include, but is not limited to, a nucleic acid sequence located at the 5′ and/or 3′ end of the oligonucleotide sequences of the present invention which hybridizes a complementary nucleic acid sequence. In one aspect of the invention, an amplification site is removed from the oligonucleotide after amplification. In another aspect of the invention, an amplification site includes one or more restriction endonuclease recognition sequences recognized by one or more restriction enzymes. In another aspect, an amplification site is heat labile and/or photo labile and is cleavable by heat or light, respectively. In yet another aspect, an amplification site is a ribonucleic acid sequence cleavable by RNase. In still another aspect, an amplification site is chemically cleavable (e.g., using acid and/or base).

As used herein, the term “restriction endonuclease recognition site” is intended to include, but is not limited to, a particular nucleic acid sequence to which one or more restriction enzymes bind, resulting in cleavage of a DNA molecule either at the restriction endonuclease recognition sequence itself, or at a sequence distal to the restriction endonuclease recognition sequence. Restriction enzymes include, but are not limited to, type I enzymes, type II enzymes, type IIS enzymes, type Ill enzymes and type IV enzymes. The REBASE database provides a comprehensive database of information about restriction enzymes, DNA methyltransferases and related proteins involved in restriction-modification. It contains both published and unpublished work with information about restriction endonuclease recognition sites and restriction endonuclease cleavage sites, isoschizomers, commercial availability, crystal and sequence data (see Roberts et al. (2005) Nucl. Acids Res. 33:D230, incorporated herein by reference in its entirety for all purposes).

In certain aspects, primers of the present invention include one or more restriction endonuclease recognition sites that enable type IIS enzymes to cleave the nucleic acid several base pairs 3′ to the restriction endonuclease recognition sequence. As used herein, the term “type IIS” refers to a restriction enzyme that cuts at a site remote from its recognition sequence. Type IIS enzymes are known to cut at a distances from their recognition sites ranging from 0 to 20 base pairs. Examples of Type Hs endonucleases include, for example, enzymes that produce a 3′ overhang, such as, for example, Bsr I, Bsm I, BstF5 I, BsrD I, Bts I, Mnl I, BciV I, Hph I, Mbo II, Eci I, Acu I, Bpm I, Mme I, BsaX I, Bcg I, Bae I, Bfi I, TspDT I, TspGW I, Taq II, Eco57 I, Eco57M I, Gsu I, Ppi I, and Psr I; enzymes that produce a 5′ overhang such as, for example, BsmA I, Ple I, Fau I, Sap I, BspM I, SfaN I, Hga I, Bvb I, Fok I, BceA I, BsmF I, Ksp632 I, Eco31 I, Esp3 I, Aar I; and enzymes that produce a blunt end, such as, for example, Mly I and Btr I. Type-Hs endonucleases are commercially available and are well known in the art (New England Biolabs, Ipswich, Mass.). Information about the recognition sites, cut sites and conditions for digestion using type Hs endonucleases may be found, for example, on the Worldwide Web at neb.com/nebecomm/enzymefindersearch bytypeIIs.asp). Restriction endonuclease sequences and restriction enzymes are well known in the art and restriction enzymes are commercially available (New England Biolabs).

Certain exemplary embodiments are directed to the use of computer software to automate design and/or interpretation of genomic spacings, repeat-discriminating SNPs and/or colors for each specific oligopaint set. Such software may be used in conjunction with individuals performing interpretation by hand or in a semi-automated fashion or combined with an automated system. In at least some embodiments, the design and/or interpretation software is implemented in a program written in the JAVA programming language. The program may be compiled into an executable that may then be run from a command prompt in the WINDOWS XP operating system. Unless specifically set forth in the claims, the invention is not limited to implementation using a specific programming language, operating system environment or hardware platform.

It is to be understood that the embodiments of the present invention which have been described are merely illustrative of some of the applications of the principles of the present invention. Numerous modifications may be made by those skilled in the art based upon the teachings presented herein without departing from the true spirit and scope of the invention. The contents of all references, patents and published patent applications cited throughout this application are hereby incorporated by reference in their entirety for all purposes.

The following examples are set forth as being representative of the present invention. These examples are not to be construed as limiting the scope of the invention as these and other equivalent embodiments will be apparent in view of the present disclosure, figures, tables and accompanying claims.

### Example I

### Converting Multi-Well Plates to Slides

A novel separable multi-well apparatus (e.g., separable multi-well plate) is provided having a base component that can be separated from a well-forming component. One advantage of such an apparatus is that the base component is processed as one processes a slide, facilitating many manipulations. One such manipulation is that is simplified is processing of samples from the wells for imaging during whole-genome screens using RNAi or small molecules. The reduction of a multi-well plate to a base component having the well-forming component removed will facilitate assays involving stains, FISH, antibodies, and the like that are applied uniformly across all samples on the base component (FIG. 1).

It is possible that separation of the base component from the well-forming component will dislodge one or more samples from the slide. In such instances, a circular cutting, heating and/or laser device is inserted into the wells to separate the samples from the sides of the wells.

### Example II

### Traditional FISH Protocol

The FISH protocol was adapted from previously published protocols and involved the following steps. Cells from log-phase cultures were adhered to either gelatin-coated (0.2%; Sigma G1393) or lysine-treated (Sigma P8920) 10-well glass slides (Erie Scientific ER208 W) for 1 to 3 hours. Slides were then gently washed with PBS (pH 7.2), fixed for 5 minutes with 4% formaldehyde in PBS (Electron Sciences 15700) at room temperature (RT), covered with a cover slip, frozen on an aluminum block (which had been pre-cooled on dry ice), freed of their cover slips, and stored in 95% ethanol at −20° C. After at least 20 minutes, slides were washed in 2×SSCT (0.3 M NaCl, 0.03 M sodium citrate, 0.1% Tween-20)/formamide (5 minutes each in 0%, 20%, 40%, and 50% formamide at RT, and 30 minutes in 50% formamide/2×SSCT at 37° C.). DNA probe in hybridization buffer was then added to the slides, covered with a cover slip, and denatured in an MJ Research PTC-200 thermocycler with an Alpha Unit™ lock Assembly block for 2 minutes at 91° C., after which slides were transferred to a humidifying chamber, incubated overnight at 37-40° C. and freed of their cover slips while being washed (30 minutes in 50% formamide/2×SSCT at 37° C., 5 minutes in 25% 2×SSCT/formamide at RT, and 3 times in 5 minutes in 2×SSCT at room temperature).

To visualize probes, slides were blocked in blocking buffer (0.1% BSA in 2×SSCT) at RT for 30 minutes, incubated with either rhodamine conjugated anti-DIG antibody (Roche 1207750) or fluorescein anti-biotin (Vector SP-3040) in blocking buffer for 1.5 hours, and washed for 1 hour in 2×SSCT, after which Vectashield with DAPI (Vecto: H-1200) was added. Cover slips were applied and sealed to the slides with nail polish.

DNA probes were synthesized according to standard protocols. P1 plasmids (Berkeley Drosophila Genome Project) containing cloned Drosophila genomic DNA corresponding to chromosomal regions 21E3-4 (abbreviated as 21E3); DS03071; 28B1-28B2 (abbreviated as 28B1); 40A2-40A3 (abbreviated as 40A2); and 69C2-69C8 (abbreviated as 69C2; DS02752) were digested with restriction enzymes (and then labeled with either Digoxigenin (DIG-Nick Translation Mix, Roche Diagnostics 1 745 816) or, for dual label experiments, biotin (BioNick™ Labeling System, Invitrogen LT18247-015)) following the manufacturers' protocols. Probe for 16E1-16E2 (abbreviated as 16E1) was synthesized from the bacterial artificial chromosome BACR17D02 RP98-17D2 (AC012163; AE003507) by nick translation/direct labeling (Vysis 32-801300) following the manufacturer's protocol. The 359-bp repeat probe was synthesized by PCR. Probes for 8C8 and 44F1 were synthesized from PCR products): eight to ten 1-1.4 kbp PCR products corresponding to genomic regions separated by approximately 1 kbp and spanning approximately 30 kbp were combined, purified, and labeled by Nick translation (Invitrogen FISH-Tag™ DNA Kit). Probes were diluted into hybridization buffer (50% formamide/2×SSCT, 10% dextran sulfate) to a final concentration of approximately 150 to 500 ng/30 μL.

Oligonucleotide probes for the AACAC and dodeca heterochromatic repeats were synthesized with either a 5′ Cy3 or Cy5 fluorescent dye (Phoenix BioTechnologies) and contained locked nucleic acid (LNA; Silahtaroglu et al. (2003) Mol. Cell Probes 17:165; Silahtaroglu et al. (2004) Cytogenet. Genome Res. 107:32) bases to increase melting temperature (AaCaCaAcAcAaCaCaAcAc (SEQ ID NO:1) and AcGgGaCcAgTaCgG (SEQ ID NO:2), for “AACAC” and “dodeca” probes, respectively, where capital letters denote LNA-modified nucleotides). An abbreviated FISH protocol was developed for LNA containing oligonucleotides: after fixation, cells were incubated for 30 minutes in 2×SSCT at 37° C., after which probe (1-100 nM in hybridization buffer) was added and slides denatured at 91° C. for 2 minutes, washed immediately in 2×SSCT for 30 minutes at 37° C., and then mounted with Vectashield.

### Example III

### High-Throughput LNA FISH Protocol

Kc₁₆₇ Cell Cultures & RNAi Preparation for 384-Well Plate Experiments

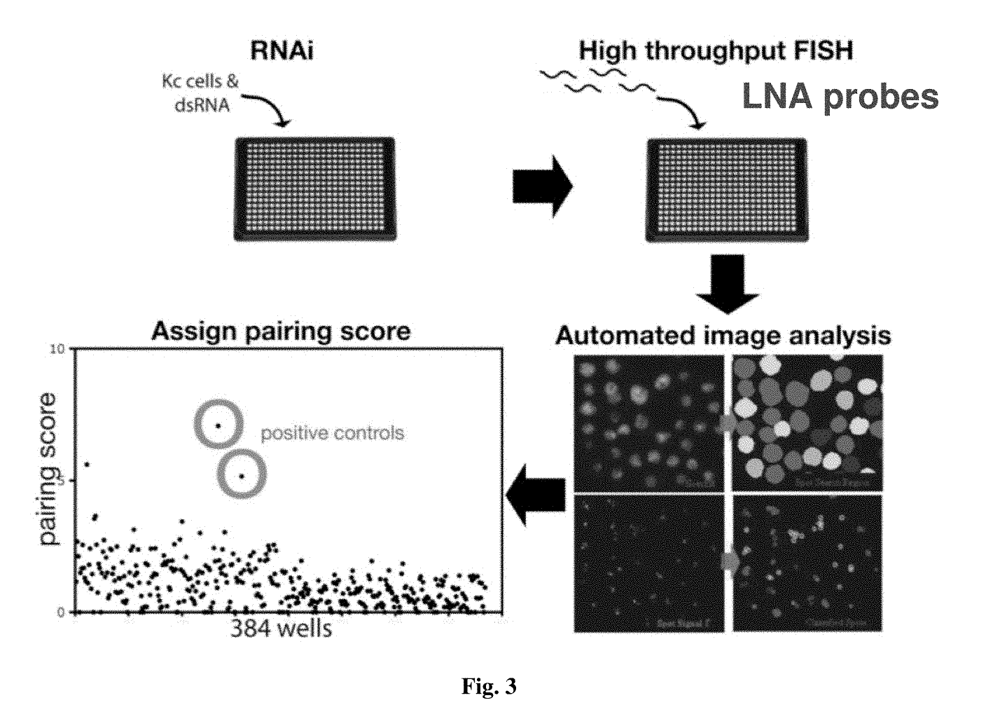

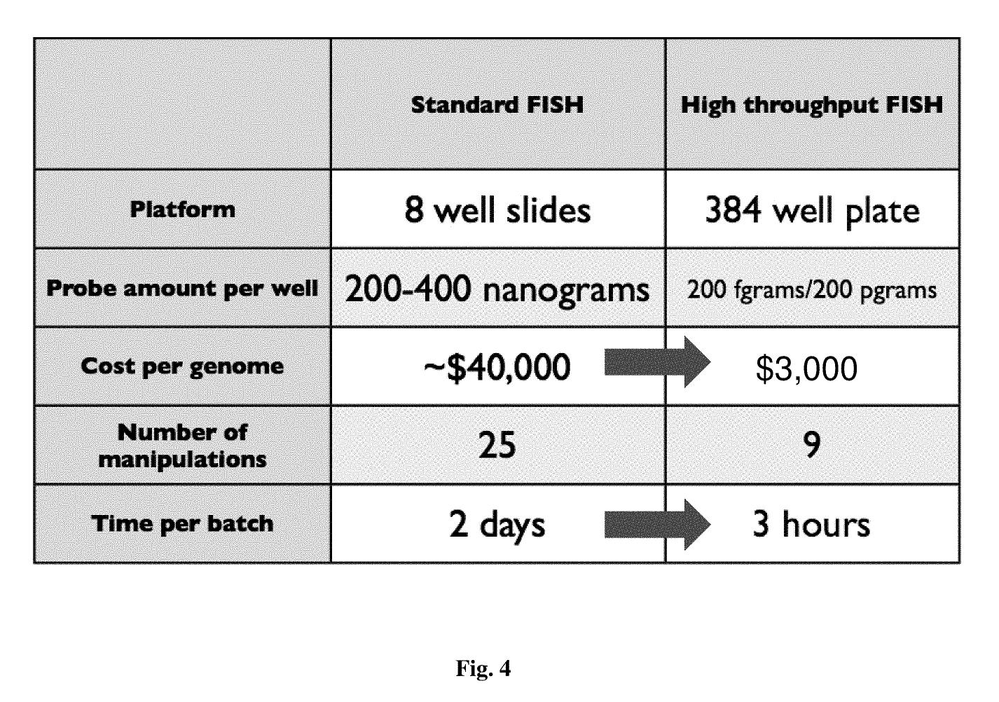

See FIGS. 3 and 4. All reagents were warmed to room temperature prior to use. A 384-well plate containing ds RNA was spun down at 1000 RPM for 2 minutes. Log-phase Kc₁₆₇ cells (grown for 3 days) were scraped from T75 cell culture flask and spun down (1000 RPM for 5 minutes). Cells were counted and diluted in FBS-free media (1-5×10⁶ cells/mL). Sterile, autoclaved Wellmate tubing was purged with sterile PBS while aluminum foil was used to keep sterile items covered. The Wellmate was primed and diluted cells were added to each well (10 μL/well). The plate was then spun (1000 RPM, 2 minutes) and incubated in a 25° incubator for 30 minutes. With freshly primed sterile tubing, regular Schneider's media was added to each well (30 μL/well) and the plate was spun (1000 RPM, 2 minutes) before placing in a 25° cell culture incubator.

Fixing and Washing of Cells

4.5 days after ds RNA treatment, the plates were removed from the incubator. The cell media was aspirated and, with a primed Wellmate, wells were quickly washed with PBS (60 μl/well), which was then immediately aspirated. Plates were incubated with 4% Formaldehyde (30 μl/well) for 5 minutes aspirated and quickly rinsed (30 μl PBS/well), then washed with 2×SSCT (80 μl/well) for 5 minutes. If plates were stored, the wells were washed again (80 μl/well) and stored sealed at 4° C. until the next step. Then, plates were washed with 50% formamide/2×SSCT (80 μl/well) for 5 minutes. The plates were sealed with an adhesive aluminum seal, pre-incubated at 91° for 3 minutes, 60° for 20 minutes, and allowed to cool to room temperature.

### LNA FISH in 384-Well Plates

Probe was prepared in Hybridization Buffer (HB): 100 μl of 100 μM H1-Cy3 Probe and 1.0 μl of 1:10 dilution of 100 μM DOD-A488 Probe were added into 10 mL HB. For probe sequences, see Table 1.

TABLE 1
LNA oligonucleotide FISH probes designed and tested in Drosophila cell culture.

| LOCATION | Probe | Repeat | | Tₘ | Number | Signal |
|---|---|---|---|---|---|---|
| (chromosome) | Name | Name | Sequence | (C.) | of repeats | quality |
| 14A-B (X) | 9.2 | 15852279 | CtCaAgAaGaTaCaAgGaCa | 78° | 42 | Good |
| | | | (SEQ ID NO: 3) | | | |
| 14A-B (X) | 9.3 | 15852279 | CcAgTgCaGaAgAaAaTcAa | 71° | 57 | Good |
| | | | (SEQ ID NO: 4) | | | |
| 5-S RNA (2R) | 5SRNA | SS-RNA | GcCcAtAgAcTgAaAtAgA | 74° | 94 | Good |
| | | repeat | (SEQ ID NO: 5) | | | |
| 39D2-39E1 | His2A | Histone | GgaCgtGgaAaaGgtGgcAaaG | 85° | 110 | Good |
| | | Complex | (SEQ ID NO: 6) | | | |
| 39D2-39E1 | His2B | Histone | AagCgcTcgAccAtcAccAgtC | 80° | 110 | Good |
| HisC | | Complex | (SEQ ID NO: 7) | | | |
| 39D2-39E1 | His1 | Histone | AaAaAgAcGgTgAaGaAaGcAt | 78° | 110 | Good/robust |
| HisC | | Complex | (SEQ ID NO: 8) | | | |
| III: Pericentric | DOD | dodeca | AcGgGaCcAgTaCgG | 85° | Unknown | Robust |
| heterochromatin | | repeat | (SEQ ID NO: 9) | | | |
| III | | | | | | |
| Transgenic | LacO1 | LacO | GtGaGcGgAtAaCaAtt | 71° | 256 | Robust |
| LacO array | | | (SEQ ID NO: 10) | | | |
| Transgenic | LacO2 | LacO | AtGtGgAaTtGtGaGcG | 75° | 256 | No signal |
| LacO array | | | (SEQ ID NO: 11) | | | |
| II-R: Pericentric | CAC | AACACₙ | AaCaCaAcAcAaCaCaAcAC | 79° | Unknown | Robust on |
| heterochromatin | | | (SEQ ID NO: 12) | | | spreads; |
| | | | | | | unclear in |
| | | | | | nucleus | |
| II-L: Pericentric | TAG | AATAGₙ | AaTaGaAtAgAaTaGaAtAG | 65° | Unknown | Robust on |
| heterochromatin | | | (SEQ ID NO: 13) | | | spreads; |
| | | | | | | unclear in |
| | | | | | | nucleus |

Each plate was aspirated and probe mix was added to each well (20 μl/well). The plates were double sealed (with clear and aluminum adhesive seals), spun (1000 RPM for 2 minutes), denatured at 91° for 20 minutes, and incubated for 1:20 hours at 44° by floating the plate in pre-warmed 44° wash buffer (50% formamide in 2×SSCT).

To wash the plate, it was submerged under the 44° wash buffer and the seal was removed, allowing the buffer to immediately wash into the wells. The plate and buffer were placed on a slow moving shaker. Buffer was vigorously “flicked” out of the wells after 5 minutes and quickly re-submerged. This was repeated twice, changed to a second clean wash buffer, and repeated three times. These wash steps were essential to reducing nuclear background fluorescence.

The plate was aspirated and washed with room temperature 50% formamide/2×SSCT (80 μl/well) for 5 minutes. 2×SSCT with Hoechst was added (30 μl/well; 30 μl Hoechst+30 mL 2×SSCT) for 5 minutes. The plate was washed with 2×SSCT (60 μl/well), let sit for 10 minutes, which was repeated. The plate was spun (1000 RPM for 2 minutes).

A comparison of standard FISH (Example II) and high-throughput LNA FISH (Example III) are set forth at Table 2.

TABLE 2
Comparison of standard FISH and LNA FISH after simplification and re-optimization.

| Standard FISH on glass slide | | | LNA FISH in 384-well plates | | |
|---|---|---|---|---|---|
| Time | | Protocol step | Time | | Protocol Step |
| 15 | m | Fix cells on glass slide | 15 | m | Fix cells on glass slide, washed with |
| | | | | | PBS |
| 30 | m | Freeze slide at −80° C., crack off cover | | | — |
| | | slip | | | |
| 30 | m | Put slide in −20° C. 95% Ethanol | | | — |
| 30 | m | Wash slide in 2xSSCT buffer (3X) | 5 | m | Wash plate with 2xSSCT buffer (1X) |
| 45 | m | Wash in 10, 20, 40, 50% | | | — |
| | | 2xSSCT/Formamide | | | |
| 30 | m | Incubate at 40° C. in 50% 2x SSCT | 20 | m | Emerged in at 44° C. in 50% |
| | | | | | 2xSSCT/Formamide |
| 5 | m | Add Probe in Hybridization Buffer (3 μL) | 5 | m | Add Fluorescent probe in |
| | | | | | Hybridization Buffer (10-20 μL) |
| Overnight | | Denature at 91° C. in slide | 1 | h | Denature at 91° C. in water bath, |
| | | Thermocycler, incubate overnight | | | incubate at 44° C. in water bath for 1 |
| | | incubated at 40° C. | | | hour |
| 45 | m | Wash at 40° C. | 45 | m | Wash at 44° C. |
| 45 | m | Washed in 50, 40, 20, 10% | | | — |
| | | 2xSSCT/Formamide | | | |
| 2 | h | Block, add fluorescent antibody | | | — |
| | | targeting probe | | | |
| 2 | h | Wash slides | | | — |
| 5 | m | Add DAPI, image | 5 | m | Add Hoechst dye, image |
| ~1.5 | days total | 8 samples per slide | ~2 | h total | 384 samples per plate |

Many steps were eliminated, including multiple wash steps. Other steps were modified to accommodate 384-well plates, such as denaturing and incubating the plates in water baths instead of on solid surfaces.

Automated Imaging

The cells were imaged with an Evotec Opera Confocal Screening Microscope (Perkin-Elmer). A 40× water immersion lens was used. Ten images per well were acquired. The plates were imaged twice, once with the Hoechst channel imaged with a confocal light source, and once with the Hoechst channel excited with a non-confocal UV lamp (no differences between the methods were apparent).

LNA FISH Targeting LacO Array in Caulobacter

FISH protocols were successfully tested using two LNA oligonucleotides probes in the bacteria Caulobacter. Two fluorescent LNA oligonucleotides were tested that target the LacO repeat and were labeled with different types of biotin 5′-DualBiotin-oligo-Cy5-3′ (Dual Biotin: two conjugated biotins, common in SAGE protocols) and 5′Cy5-oligo-BioTEG (BioTEG: biotin conjugated with a spacer). Despite heavy modifications, both probes label efficiently as demonstrated by co-localization with the CFP:LacI, a protein that also binds the LacO array. As for optimization, a low concentration was needed (<1 nM). Of note, this protocol lacks a 91° denaturation step:

1) 1.6% formaldehyde for 15′, fix and wash
2) Dry cells on slide with lysozyme
3) 37° C. incubation with probe, 30′
4) 37° C. wash, 30′
5) Mounted with DAPI containing buffer

### Example IV

### FISH Probes for Improved High-Throughput FISH

The use of oligonucleotide paints (Oligopaints) enables high-throughput FISH analyses without the need for costly LNAs, thereby making it possible for the first time to perform whole genome RNAi and/or small molecule driven screens for genes involved in chromosome positioning. Oligopaints provide key advances in the clinical setting, as they are an attractive, low-cost, higher resolution alternative to stains that are currently available for karyotyping in disease diagnosis and prenatal care.

Commercially available chromosome paints are made from FACS-sorted chromosomes and, at higher resolutions, from chromosomes that have been dissected into smaller fragments via bacterial artificial chromosomes or other cloning vectors. While these paints have contributed remarkably to the ability to visualize chromosomes via FISH, they are costly, often prohibitively so, and limited in resolution (Worldwide Web: chrombios.com; openbiosystems.com/FISHprobes/Starfish/Human/Multicolor/; and metasystems.de/www2/index.php?option=com_djcatalog&view=show&cid=20&Itemid=61&layout=default).

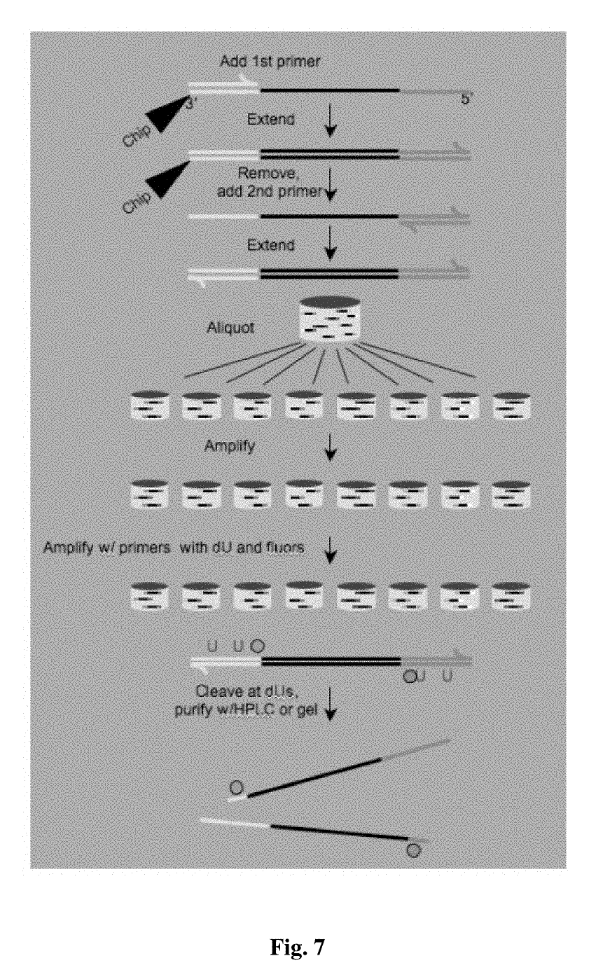

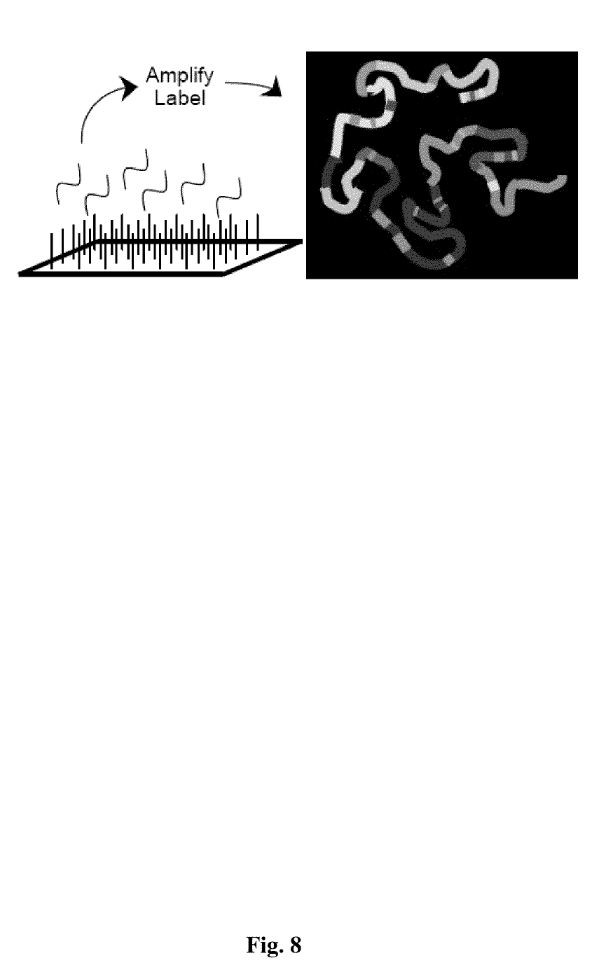

In contrast, custom-designed paints are described herein that are generated via PCR amplification of genomic sequences synthesized on arrays (FIG. 8). In particular, PCR amplification of 60-bp oligomers containing 32 bp of genomic sequence flanked by 14-bp primer sequences, wherein the totality of genomic sequence represents 20-40% of the non-repetitive portion of a genome is performed. FIG. 8 schematically depicts a chromosome that has been Oligopainted (finer bands not shown). FIG. 7 describes one protocol for recovering oligomers from arrays and then, by aliquoting and PCR, producing pools of products which, when labelled with fluors and used as probes in FISH, decorate chromosomes with custom-designed bands of colors.

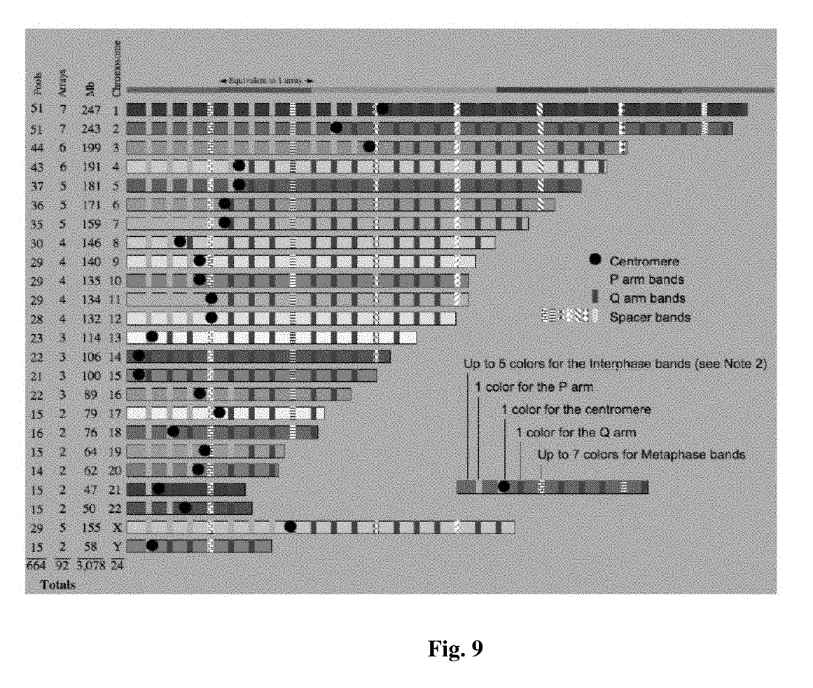

FIG. 9 depicts one strategy by which the 24 chromosomes of the human genome are differentially colored with a base color consisting of a mix of five primary colors and a series of color-coded bands staggered along the p and q arms. Patterns tailored to the needs of the researcher are generated by computer algorithms that select 32-bp sequences and then associated with a judicious selection of primer sequences such that their amplification with primers carrying dyes, followed by FISH, creates distinct bands.

Arrays representing 55 different primers pairs were created that flanked 20% of the non-repetitive regions of Chromosome 19, the Drosophila×chromosome, and C. elegans×chromosome. Importantly, 43 of the 55 primer pairs produced products (FIG. 5). The products are sequenced to assess how well they represent the genome in terms of sequence fidelity and coverage. Without intending to be bound by scientific theory, because each band can contain hundreds or thousands of different 32-bp targets, this technology should tolerate significant sequence degeneracy in the probe.

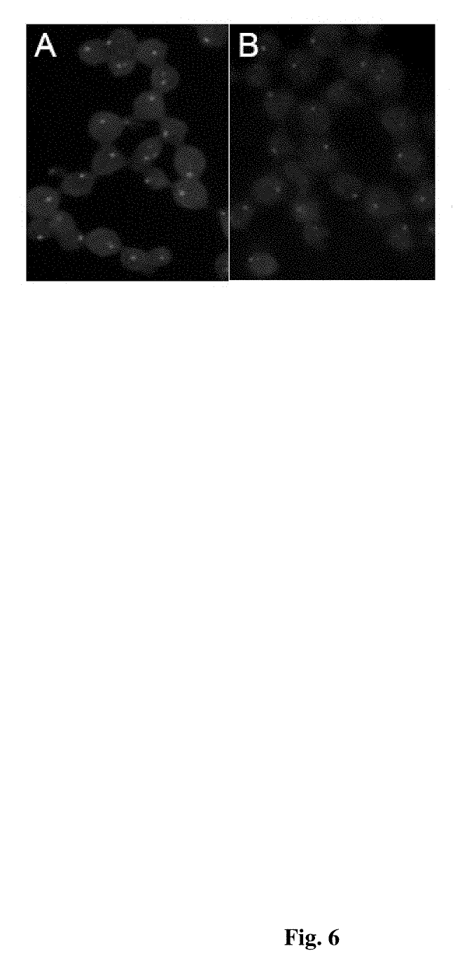

A challenging aspect of this technology development is the determination of the conditions under which 60-bp oligomers, containing only 32-bp of homology to their targets, will succeed as FISH probes. Results obtained thus far have been promising. Signals have been obtained using Drosophila cells and single stranded 60-bp probes synthesized with a DNA synthesizer and homologous to approximately 100 copies of a 32-bp target (FIG. 5). Signals have also been obtained using the same target using synthesized 32-bp probes using a 384-well FISH protocol (FIG. 6). These data demonstrate that 32 bp of homology is sufficient for generating FISH signals. Trials were being conducted with synthesized double stranded 60- and 32-bp probes as well as probes derived from the array (FIG. 7).

### Example V

### Increasing Nucleic Acid Affinity Via Nucleases

Without intending to be bound by scientific theory, it was hypothesized that the nicked 60-bp of Example IV worked as a FISH probe, while a ds 60-bp of Example IV did not, because the ds 60-bp oligonucleotide sequence was constrained by primer homology at both ends. A nick removed one of these constraints, potentially resulting in a conformation more favorable to the finding of its genomic target. A slight relaxation of genomic DNA, catalyzed e.g., by a limiting DNase I or MNase digestion (or digestion with any nicking or cutting enzyme, such as a restriction enzyme) is performed after fixation, to create a more permissive environment for the hybridization of our probes to allow use of ds 60-bp oligonucleotide sequences. The use of nucleases increases the affinity of any nucleic acid-nucleic acid hybridization (thus increasing probe-target binding), where the constraints of one or more nucleic acid molecules may inhibit binding. These methods are particularly useful for FISH with oligonucleotide paints. In certain aspects, the binding of nucleic acids to non-nucleic acid substrates is promoted.

### Example VI

### Targeting Endonucleases to Probes

Methods of releasing 3′ primer sequences from PCR-derived probes consisting of genomic sequence flanked by primers are provided. These methods are particularly useful with FISH. Accordingly, in certain aspects, fluorescent tags are introduced via the primers.

Strategy 1

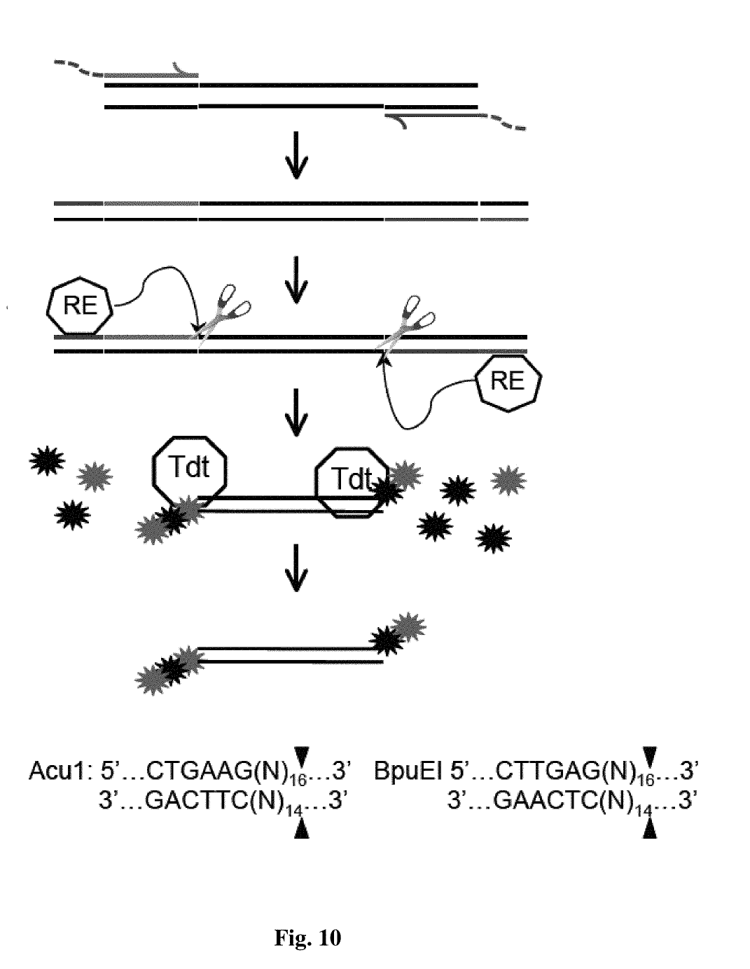

In a first embodiment, methods of releasing 5′ and 3′ primer sequences using restriction enzymes that generate double stranded cuts (e.g., type IIS restriction enzymes) are provided (FIG. 10). The first step of this method includes performing PCR using ‘touch-up’ primers that add restriction enzyme sites specific for enzymes that cause a double-stranded break in the target nucleic acid molecule (e.g., type IIS restrictions sites (e.g., AcuI, BpuE1, etc.)). The second step involves restriction digestion of PCR products to produce ds 32-mers with both primer sequences removed, followed by the addition of dTTP and Cy3-dUTP using, e.g., terminal transferase (step three). The fourth step is to clean up and concentrate the labeled probe using columns.

### Strategy 2

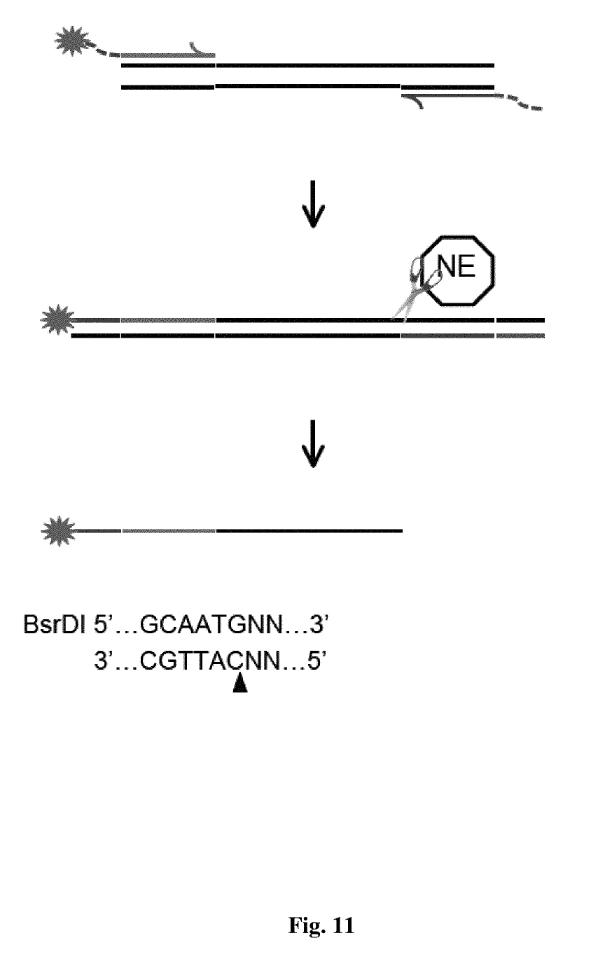

In a second embodiment, methods of releasing 3′ primer sequences are provided, e.g., using a 5′ end label with a nicking endonuclease (FIG. 11). The first step of this method is to perform PCR with a 5′ end labelled primer (e.g., using a primer having a fluorescent label bound thereto) (depicted in red in FIG. 11) and, optionally, an unlabeled amplification primer (depicted in blue in FIG. 11). This is depicted for just one strand, but it could be performed on two strands. In certain aspects, PCR is performed with Cy3-dUTP to boost signal. 0 bp, 4 bp and 7 bp touch up can be used. The second step includes digestion with a restriction enzyme that cuts one strand (e.g., a bottom strand nicking endonuclease (e.g., BsrDI)), followed by denaturation. This generates a single stranded nucleic acid sequence having a fluorescent label attached thereto, but lacking the 3′ primer sequence. The third step includes cleaning up and concentrating the probe. In certain aspects, cold primer that is complementary to the unlabeled strand is used to purify the labelled strands. In other aspects, gel purification of the labelled strands is performed.

### Example VII

### High-Throughput FISH Methods

Method 1

Oligopaints are provided for performing high-throughput FISH. This method combines restriction digestion of the Oligopaints using Strategy 1 and/or Strategy 2 of Example VI. This method optionally utilizes one or more multi-well apparatuses having removable wells as described herein (e.g., in Example I). This method decreases the cost of FISH and is particularly useful for analyzing nucleic acid sequences that are present in a genome in low or single copy numbers.

Method 2

Probes having homology to one or more nucleic acid sequences of interest (e.g., genomic sequences of interest) are provided for performing high-throughput FISH. This method optionally avoids the use of Oligopaints. This method combines restriction digestion of the probes using Strategy 1 and/or Strategy 2 of Example VI. This method optionally utilizes one or more multi-well apparatuses having removable wells as described herein (e.g., in Example I). This method decreases the cost of FISH and is particularly useful for analyzing moderately repetitive elements and/or highly repetitive elements of a genome.

### Example VIII

### Genes that Control Homolog Pairing in Somatic Cells

Background and Significance

Methods and compositions to identify factors that mediate somatic homolog pairing, as they are the key for efficient homologous gene replacement and, thus, technologies for gene therapies, are provided. The inability to achieve efficient rates of homologous gene replacement in somatic cells is the single greatest reason that we do not have practical technologies for gene therapy. Lack of such technologies equally hinders basic research using any organism more complex than viruses, bacteria, or fungi. Millions of dollars are spent each year coaxing two pieces of DNA to exchange genetic information, but have yielded no fully satisfactory protocols. Instead, researchers using human cells, mice, flies, worms, and plants either contend with time-consuming methods or resort to inserting DNA randomly into the genome, the latter route often generating confounding and, in the case of humans, potentially dangerous complications.

The underlying problem is the tremendous lack of understanding of the process that pairs a chromosomal segment with its homolog. While many of the enzymes that mediate the recombination of DNA after pairing has occurred are known, the mechanism by which two pieces of DNA find each other remains essentially unknown. In one aspect, factors that mediate pairing are identified.

Aspects of the invention provide a newly developed protocol that enables FISH to be carried out on cell cultures in a parallel (e.g., high-throughput) format (e.g., using 384-well plates). As such, the protocol permits, for the first time, a high-throughput whole genome FISH- and RNAi-based screen for genes that are important for pairing. A newly established Drosophila cell culture-based experimental system is used for the analysis of somatic pairing (Williams et al. (2007) Genetics 177:31).

Current protocols for homologous gene replacement in mammalian cells tend to hover around 10⁵ to 10⁷ events/cell, far too low for their routine use in a medical setting (Capecchi (2005) Nat. Genet. 6:507). While many studies have aimed to improve on these frequencies (Capecchi, Supra; Yang and Seed (2003) Nat. Biotech. 21:447), much less effort has focused on the process of homolog pairing, which must occur before recombination takes place. This paucity of studies is due primarily to the fact that pairing rarely occurs in mammalian somatic cells, precluding use of these cells for the analysis of pairing.

One approach provided herein is to focus on the single model organism, Drosophila, that supports extensive homolog pairing in somatic cells. In fact, Drosophila pairs its homologs in virtually all cells throughout essentially all of development, making it an ideal experimental system for the analysis of homolog pairing (McKee (2004) Biochim. Biophys. Acta 1677:165). Thus far, Drosophila has been used to clarify how pairing can alter the way genes are expressed (Lee and Wu (2007) Genetics 174:1867) and demonstrated Drosophila cell culture to be a viable system for the analysis of homolog pairing (Williams et al., Supra). Specifically, it has been shown that pairing in cell culture is genome-wide and impervious to cell type, culture history, or cell cycle changes. As proof of principle, the Drosophila system has also been used it to identify topoisomerase II as a factor which, when disrupted, can lead to the unpairing of homologs (Williams et al., Supra). Finally, as described further herein, this system was used to adapt FISH for use in 384-well plates, making it possible to carry out whole-genome RNAi-based screens for genes important for homolog pairing. Importantly, a pilot run covering one ninth of the Drosophila genome easily pulled out several strong candidate genes even though the most rigorous standards were used for assessing the data.

Drosophila is used in cell culture to conduct a whole genome FISH- and RNAi-based screen for genes that are involved in somatic homolog pairing. Candidate genes are those which, when disrupted by RNAi, cause homologs to unpair. In certain aspects, this screen will be performed using Oligopaints specific for single copy regions of the genome.

Without intending to be bound by scientific theory, it is proposed that the paired state of homologs is the default state and that mammalian somatic cells actively prevent pairing (Lee and Wu, Supra). As such, we will use Oligopaints are used with mammalian cells to conduct whole genome RNAi-driven screens, wherein candidate genes are identified as those which, when disrupted, reduce the volume of the nucleus occupied by the target chromosome.

Data collection and analyses are automated for the screens, while candidate genes are further characterized on an individual basis. Note that, in certain aspects, the screens are also conducted with small molecules.

1.-24. (canceled)

25. A method for performing fluorescence in situ hybridization (FISH) comprising:
providing a plurality of biological samples including a nuclear membrane containing a target chromosome sequence on a multi-well plate having a well-forming component separably attached to a base component;
contacting the plurality of biological samples with an oligonucleotide paint having a fluorescent label attached thereto and having a length within a range of from 4 to 36 nucleotides, wherein the oligonucleotide paint crosses the nuclear membrane;
allowing the oligonucleotide paint to bind to the target chromosome sequence;
separating the samples from the sides of the wells of the well-forming component to prevent sample dislodging from the well-forming component;
removing the well-forming component from the base component; and
detecting binding of the oligonucleotide paint on the base component by imaging the base component.

26. The method of claim 25, wherein a plurality of oligonucleotide paints is used.

27. The method of claim 25, wherein the oligonucleotide paint crosses a cell membrane.

28. The method of claim 25, wherein the oligonucleotide paint binds to the plurality of biological samples by hybridizing to the target chromosome sequence.

29. The method of claim 28, wherein the target chromosome sequence is a low copy number nucleic acid sequence.

30. The method of claim 29, wherein the nucleic acid sequence is a single copy number nucleic acid sequence.

31. The method of claim 25, wherein the multi-well plate is a 384-well plate.

32. A method for performing fluorescence in situ hybridization (FISH) comprising:
providing a plurality of biological samples including a nuclear membrane into wells of a separable multi-well apparatus having a well-forming component and a base component;
contacting the biological samples with an oligonucleotide paint having a fluorescent label attached thereto, wherein the oligonucleotide paint crosses the nuclear membrane;
allowing the oligonucleotide paint to bind to the biological samples;
separating the samples from the sides of the wells of the well-forming component to prevent sample dislodging from the well-forming component;
removing the well-forming component from the base component; and
contacting the base component with one or more reagents.

33. The method of claim 32, wherein the oligonucleotide paint lacks a primer binding sequence at the 3′ end.

34. The method of claim 33, wherein the primer binding sequence at the 3′ end is lacking as having been removed from the oligonucleotide paint by contacting the oligonucleotide paint with a nicking endonuclease.

35. The method of claim 33, wherein the oligonucleotide paint having the primer binding sequence at the 3′ end removed binds the biological sample with a greater affinity when compared to an oligonucleotide paint having any primer binding sequence at the 3′ end present.

36. The method of claim 32, wherein the oligonucleotide paint lacks a primer binding sequence at the 3′ end and primer binding sequence at the 5′ end.

37. The method of claim 36, wherein the primer binding sequences at the 3′ and the 5′ ends are lacking as having been removed from the oligonucleotide paint by contacting the oligonucleotide paint with a type IIS restriction enzyme.

38. The method of claim 36, wherein the oligonucleotide paint having the primer binding sequences at the 3′ and the 5′ ends removed binds the biological sample with a greater affinity when compared to an oligonucleotide paint having 3′ and 5′ primer sequences present.

39. The method of claim 36, wherein the fluorescent label is attached to the oligonucleotide paint using terminal transferase.

40. A method for performing FISH comprising:
providing a plurality of biological samples including chromosomal DNA into wells of a multi-well plate having a well-forming component separably attached to a base component;
contacting the chromosomal DNA with an enzyme that cleaves the chromosomal DNA in a manner to limit digestion;
contacting the chromosomal DNA with an oligonucleotide paint having a fluorescent label bound thereto;
allowing the oligonucleotide paint to bind to the chromosomal DNA;
separating the samples from the sides of the wells of the well-forming component to prevent sample dislodging from the well-forming component;
removing the well-forming component from the base component; and
detecting binding of the oligonucleotide paint to the chromosomal DNA.

41. The method of claim 40, wherein the enzyme that cleaves DNA is one or both of a nuclease and a restriction enzyme.

42. The method of claim 41, wherein the nuclease is one or both of DNase I and micrococcal nuclease.

43. The method of claim 40, wherein the oligonucleotide paint binds to the biological sample by hybridizing to low or single copy genomic DNA.

44. The method of claim 40, wherein the oligonucleotide paint is of 32 nucleotides of genomic sequence.

45. The method of claim 40, further including a plurality of oligonucleotide paints and wherein the plurality of oligonucleotide paints include a totality of a genomic sequence representing 20-40% of a non-repetitive portion of a genome.

46. The method of claim 40, wherein the oligonucleotide paint includes a synthesized genomic sequence.

47. The method of claim 40, further including a plurality of oligonucleotide paints and wherein the plurality of oligonucleotide paints includes 32 nucleotides of synthesized genomic sequence representing 20-40% of a non-repetitive portion of a genome.

48. The method of claim 40, wherein the oligonucleotide paint has a length within a range of from 6 to 30 nucleotides.

49. The method of claim 40, wherein the oligonucleotide paint has a length within a range of from 8 to 20 nucleotides.

50. The method of claim 40, further comprising detecting binding of the oligonucleotide paint on the base component.

51. The method of claim 40, further comprising detecting binding of the oligonucleotide paint on the base component by imaging the base component.

52. A method for performing fluorescence in situ hybridization (FISH) comprising:
providing a plurality of somatic samples on a multi-well plate having a well-forming component separably attached to a base component;
contacting the plurality of somatic samples with an oligonucleotide paint having a fluorescent label attached thereto and having a length within a range of from 4 to 36 nucleotides;
allowing the oligonucleotide paint to bind to the plurality of somatic samples;
separating the samples from the sides of the wells of the well-forming component to prevent sample dislodging from the well-forming component;
removing the well-forming component from the base component; and
detecting binding of the oligonucleotide paint on the base component by imaging the base component.

53. A method for performing fluorescence in situ hybridization (FISH) comprising:
providing a plurality of somatic samples into wells of a separable multi-well apparatus having a well-forming component and a base component;
contacting the somatic samples with an oligonucleotide paint having a fluorescent label attached thereto;
allowing the oligonucleotide paint to bind to the somatic samples;
separating the samples from the sides of the wells of the well-forming component to prevent sample dislodging from the well-forming component;
removing the well-forming component from the base component; and
detecting binding of the oligonucleotide paint.

54. The method of claim 53, wherein the oligonucleotide paint lacks a primer binding sequence at the 3′ end.

55. The method of claim 53, wherein the oligonucleotide paint lacks a primer binding sequence at the 3′ end and primer binding sequence at the 5′ end.

56. A method for performing FISH comprising:
providing a plurality of somatic samples including chromosomal DNA into wells of a multi-well plate having a well-forming component separably attached to a base component;
contacting the chromosomal DNA with an enzyme that cleaves the chromosomal DNA in a manner to limit digestion;
contacting the chromosomal DNA with an oligonucleotide paint having a fluorescent label bound thereto;
allowing the oligonucleotide paint to bind to the chromosomal DNA;
separating the samples from the sides of the wells of the well-forming component to prevent sample dislodging from the well-forming component;
removing the well-forming component from the base component; and
detecting binding of the oligonucleotide paint to the chromosomal DNA.

57. A method for performing fluorescence in situ hybridization (FISH) comprising:
providing a plurality of somatic samples on a multi-well plate having a well-forming component separably attached to a base component;
contacting the plurality of somatic samples with amplicons of oligonucleotide paints having primer sequences removed and with each amplicon of oligonucleotide paints having a fluorescent label attached thereto and having a length within a range of from 4 to 36 nucleotides;
allowing the amplicons of oligonucleotide paints to bind to the plurality of somatic samples;
separating the samples from the sides of the wells of the well-forming component to prevent sample dislodging from the well-forming component;
removing the well-forming component from the base component; and
detecting binding of the amplicons of oligonucleotide paints on the base component by imaging the base component.
